(19) United States
(12) Patent Application Publication
(10) Pub. No.: US 2023/0107458 A1
(43) Pub. Date: Apr. 6, 2023

(54) EMULSIFYING AND TEXTURING COMPOSITION BASED ON STARCHES AND GUMS, FOR COSMETICS

(71) Applicants: ROQUETTE FRERES, Lestrem (FR); ALLIANCE GUM & INDUSTRIES, CORMEIL-LES EN PARISIS (FR)

(72) Inventors: Florence LAMOTTE D'INCAMPS, NANTERRE (FR); Géraldine LOUVET-POMMIER, LACHELLE (FR); Léon MENTINK, LILLE (FR); Sophie PIOT, PARIS (FR)

(21) Appl. No.: 17/907,064

(22) PCT Filed: Mar. 23, 2021
(86) PCT No.: PCT/FR2021/050484

§ 371 Date: Sep. 22, 2022

(30) Foreign Application Priority Data
Mar. 23, 2020 (FR) FR20 02819
Dec. 2, 2020 (FR) FR2012560

(51) Int. Cl. A61K 8/73; A61K 8/06; A61Q 19/00

(52) U.S. Cl. CPC A61K 8/732; A61K 8/06; A61K 8/73; A61Q 19/00; A61K 2800/48; A61K 2800/10

(57) ABSTRACT

The object of the present application is a solid composition for use in cosmetics, comprising at least one modified starch carrying at least one hydrophobic and/or amphiphilic functional group, at least one modified starch carrying at least one hydrophilic functional group, at least one gum of microbial or fungal origin, and at least two plant-based gums. Such a composition has emulsifying and texturing properties.

### TECHNICAL FIELD

The present application falls within the field of emulsifying cosmetic compositions for preparing an oil-in-water emulsion, stabilizing said emulsion, and providing this emulsion with a sensory profile ranging from a fluid milk to a thick cream, and a transformation texture.

### PRIOR ART

Patent US 2014/0287128 by Nisshion Oillio discloses the use of a thickening modified starch, an emulsifying modified starch, and a thickening polysaccharide selected from among plant-based gums, for preparing food seasonings in the form of an oil-in-water emulsion. This patent does not mention any use in cosmetics, and a fortiori with respect to any notion of a sensory profile for topical use.

Octenyl succinate modified starches have been widely known for their emulsifying properties since the 1950s, when National Starch filed their application U.S. Pat. No. 2,661,349. Many patent applications have subsequently been filed for improvements to these types of modified starches, in particular for methods associating a modification of the structure of the starch granules and anhydroglucose polymers that make up the starch, for example, by the action of enzymes, or of hydro-thermal treatments such as gelatinization, or dextrinification.

Starches modified, for example, by an acetyl functional group, such as acetylated starches or starch acetates, are also widely known for their texturing and thickening property. Gums of microbial or plant-based origin are also known for their texturing and thickening or gelling property. However, the synergy developed by the particular emulsifying and texturing solid composition selected by the applicant has never been disclosed, and a fortiori the transformation texture permitted by said solid composition also has never been disclosed.

### DESCRIPTION OF THE EMBODIMENTS

A first object of the present application is a solid composition comprising, or even made up of:
at least one starchy emulsifier or emulsifier of starchy origin,
at least one thickening starch,
at least one gum of microbial origin,
and at least two plant-based gums.

A second object of the present application is an oil-in-water emulsion comprising, or even made up of:
at least one starchy emulsifier or emulsifier of starchy origin,
at least one thickening starch,
at least one gum of microbial origin,
at least two plant-based gums,
and at least one oil.

A third object of the present application is the use of a solid composition that is the subject matter of the application for preparing oil-in-water emulsions for use in cosmetics, selected from among skin care, hair care or coloring, oral care or oral hygiene, hygiene, makeup, or perfume products.

A fourth object of the present application is a method for preparing an oil-in-water emulsion comprising a step of emulsifying an oil in an aqueous phase, in which the solid composition that is the subject matter of the application has been previously dispersed or solubilized.

Solid Composition:

The solid composition that is the subject matter of the present application comprises, or is made up of:
at least one starchy emulsifier or emulsifier of starchy origin,
at least one thickening starch,
at least one gum of microbial origin,
and at least two plant-based gums.

The applicant understands a “solid composition” to be a powdery or powderous form, in the form of a set of divided or agglomerated solid particles, or a composition made solid by pressing or compacting one or more powders. The size of said solid composition ranges from approximately 1 micrometer to several hundred micrometers, for example, 10 microns to 500 microns, or from 20 microns to 300 microns, and generally from 40 microns to 200 microns. The morphology of the particles can be regular, such as spheres, or irregular and angled, or a combination of different morphologies. The water content of the solid form is less than or equal to 30% by weight, relative to the total weight of the solid composition, or less than or equal to 20% by weight, or less than or equal to 15% by weight, or less than or equal to 10% by weight, or less than or equal to 5% by weight. The fraction of the water-soluble solid composition at 20° C. can be greater than or equal to 5% by weight relative to the total weight of the solid composition, or greater than or equal to 25% by weight, or greater than or equal to 50% by weight, or greater than or equal to 60% by weight, or greater than or equal to 75% by weight.

The solid composition comprises at least one starchy emulsifier or emulsifier of starchy origin. According to one embodiment, said at least one starchy emulsifier or emulsifier of starchy origin is a starch functionalized by at least one amphiphilic group selected from among an octenyl succinate granular starch, or an octenyl succinate modified pregelatinized starch, or an octenyl succinate modified gelatinized starch, or an octenyl succinate functionalized dextrin, or an octenyl succinate functionalized maltodextrin, or the mixtures thereof.

The solid composition comprises at least one thickening starch. According to one embodiment, said at least one thickening starch is selected from among stabilized starches, preferentially acetylated starches, hydroxypropylated starches, hydroxyethylated starches, or more preferentially from among pregelatinized and acetylated starches, or pregelatinized and hydroxypropylated starches, most preferentially from among pregelatinized and acetylated starches, or the mixtures thereof.

The solid composition comprises at least one gum of microbial origin. According to one embodiment, said at least one gum of microbial origin is selected from among xanthan gum, gellan gum, dextran gum, scleroglucan gum, beta-glucan gum, or the derivatives and mixtures thereof.

The solid composition comprises at least two plant-based gums. According to one embodiment, said at least two plant-based gums are selected from among galactomannans, glucomannans, galactans, alginates, preferentially from among guar gum, tara gum, locust bean gum, cassia gum, fenugreek gum, konjac gum, arabic gum, adragant gum, karaya gum, and most preferentially are guar gum and tara gum.

According to one embodiment, the solid composition comprises, or consists of, as mass percentages relative to the total weight of said solid composition:
from 20% to 60% of at least one starchy emulsifier or emulsifier of starchy origin,
from 20% to 60% of at least one thickening starch,
from 0.5% to 10% of at least one gum of microbial origin,
and from 2% to 45% of at least two plant-based gums.

In the case whereby the solid composition is made up of said mass percentages of said components, then said components are selected so that the sum thereof is equal to 100%.

According to another embodiment, said at least two plant-based gums are guar gum and tara gum. According to a variant of this embodiment, the mass proportions of the two plant-based gums, relative to the total weight of the solid composition, are:
from 1% to 30% of guar gum, or from 2% to 25%, or from 8% to 20%, or from 10% to 20%,
and from 1% to 15% of tara gum, or from 2% to 10%, or from 3% to 8%.

According to another embodiment, the solid composition comprises, or consists of, as mass percentages relative to the total weight of said solid composition:
from 20% to 60% of at least one starchy emulsifier or emulsifier of starchy origin,
from 20% to 60% of at least one thickening starch,
from 0.5% to 10% of at least one gum of microbial origin,
from 1% to 30% of guar gum,
and from 1% to 15% of tara gum.

According to another embodiment, the solid composition comprises, or consists of, as mass percentages relative to the total weight of said solid composition:
from 30% to 50% of at least one starchy emulsifier or emulsifier of starchy origin,
from 30% to 50% of at least one thickening starch,
from 0.75% to 7% of at least one gum of microbial origin,
from 2% to 25% of guar gum,
and from 2% to 10% of tara gum.

According to another embodiment, the solid composition comprises, or consists of, as mass percentages relative to the total weight of said solid composition:
from 35% to 45% of at least one starchy emulsifier or emulsifier of starchy origin,
from 35% to 45% of at least one thickening starch,
from 1% to 4% of at least one gum of microbial origin,
from 8% to 20% of guar gum,
and from 3% to 8% of tara gum.

According to another embodiment, the solid composition comprises, or consists of, as mass percentages relative to the total weight of said solid composition:
from 35% to 45% of at least one starchy emulsifier or emulsifier of starchy origin,
from 35% to 45% of at least one thickening starch,
from 1% to 4% of at least one gum of microbial origin,
from 10% to 18% of guar gum,
and from 3% to 8% of tara gum.

Thickening Starches:

The thickening starches that are useful in the invention can originate from any botanical origin, in particular wheat, corn, potato, legumes such as peas, rice, beans, broad beans. They can be granular as in the natural or pregelatinized state. Preferentially, they are selected from among pregelatinized starches, hydrolyzed starches, enzymatically treated starches, modified starches, modified dextrins.

According to one embodiment, the thickening starches are modified starches selected from among stabilized starches, preferentially from among acetylated starches, hydroxypropylated starches, hydroxyethylated starches; or from among pregelatinized and stabilized starches, preferentially from among pregelatinized and acetylated starches, pregelatinized and hydroxypropylated starches, most preferentially from among pregelatinized and acetylated starches, or the mixtures thereof.

Pregelatinized Starch:

Within the meaning of the invention, “pregelatinized starch” refers to a starch that has been made “water-soluble”, in other words a starch having, at 20° C. and under mechanical stirring for 24 hours, a soluble fraction in demineralized water that is at least equal to 5% by weight. This soluble fraction is preferably greater than 20% by weight, or more preferentially greater than 50% by weight, or most preferentially greater than or equal to 70%. Of course, the water-soluble starch can be fully soluble in demineralized water, with the soluble fraction then being greater than 90%, and can be close to 100%.

The water-soluble starch preferably has a low water content, generally less than 10%, in particular less than 5% by weight.

The pregelatinized starches are generally prepared by thermal, chemical or mechanical techniques capable of causing the starch granules to swell so that they become soluble in cold water, in particular by virtue of the release of the starchy chains forming said granules. Preferred techniques are steam cooking, jet-cooker cooking, drum cooking, cooking in mixer and/or extruder systems and then drying, for example, in an oven, by hot air on a fluidized bed, rotary drum cooking, atomization, extrusion or freeze-drying. Such starches generally exhibit solubility in demineralized water at 20° C. of more than 5% and more generally range between 10 and 100% and a rate of starch crystallinity of less than 15%, generally less than 5% and most often less than 1%, or even zero. By way of an example, the products manufactured and marketed by the Applicant under the trade name PREGEFLO® can be cited.

The pregelatinized starch also can be made up of a starch that has partially preserved its original granular form, obtained by atomization cooking, generally known as GCWS (Granular Cold Water Soluble) starch.

Hydrolyzed Starch:

“Hydrolyzed starch” is understood to mean a starch that has undergone enzymatic hydrolysis or partial chemical hydrolysis, by acid, basic or by oxidation means, resulting in a reduction in the molecular weight of the starch. Examples of low hydrolyzed starches are fluidized starches, and highly hydrolyzed starches are maltodextrins.

Dextrins:

“Dextrin” is understood to mean a starch in the form of granules that have undergone a hydro-thermal modification of their granular structure or of their intermolecular or intramolecular arrangement, by a thermal, physical or chemical action, or by a combination of these actions. The dextrins, in particular those most transformed and commonly called yellow dextrins, will be preferred within the scope of the present invention due to their beneficial solubility and stability.

Stabilized Starch:

The term “modified starch” refers to a starch having undergone a chemical treatment selected from among crosslinking, oxidation, stabilization, functionalization, or a combination of at least two of these modifications.

“Stabilized starch” is understood to mean starches that have undergone one or more of the chemical treatment(s) that are known to a person skilled in the art in order to slow down or halt the retrogradation of the starch. The stabilization is obtained by substituting hydroxyl functional groups of the starch, by esterification or etherification. It also can be obtained by oxidation. These stabilization treatments are in particular hydroxypropylation, hydroxyethylation, acetylation, phosphatation, oxidation, cationization, or carboxymethylation. According to the present invention an acetylated, or hydroxypropylated, or hydroxyethylated, preferentially an acetylated starch, is preferred. Such a stabilized starch can have a soluble fraction as defined hereinbefore that is greater than 5%, preferably greater than 10%, more preferably greater than 50%. A stabilized starch thus advantageously has the ability to thicken, until gelling, the water by simple dispersion in cold water and to yield thickened solutions, or gels, that are very stable over time, that is without evolving towards retrogradation when stored for several weeks at room temperature.

The stabilization can be particularly obtained by acetylation, in the aqueous phase, of acetic anhydride, of mixed anhydrides, by hydroxypropylation in the milk phase or in the adhesive phase, by phosphatation. These stabilized starches can have a degree of substitution ranging between 0.01 and 3, and more preferably ranging between 0.05 and 1. Preferably, the starch modification or functionalization reagents are of renewable origin.

When the stabilization is obtained by esterification, it can be achieved by employing an organic acid anhydride other than acetic anhydride, or an organic acid other than acetic acid, or a mixed anhydride, or an organic acid chloride or any mixture of these products. These products can be selected, for example, from among acids having from 1 to 24 saturated or unsaturated carbons, and more specifically from among formic acid, propionic acid, butyric acid, valeric acid, hexanoic acid, heptanoic acid, pelargonic acid, octanoic acid, decanoic acid, undecanoic acid, lauric acid, myristic acid, palmitic acid, oleic acid, stearic acid, the anhydrides of these acids, the mixed anhydrides of these acids, and any mixtures of these products.

The stabilized starch can also be a stabilized and hydrolyzed starch.

According to one embodiment, the stabilized starch is an acetylated starch, or a hydroxypropylated starch, or a hydroxyethyl starch, or a starch having undergone at least two of the chemical substitutions selected from among acetylation, hydroxypropylation, hydroxyethylation. According to one embodiment, the stabilized starch is an acetylated starch.

According to one embodiment, the stabilized starch is not crosslinked.

According to another embodiment, the stabilized starch is an acetylated waxy corn starch, or a pregelatinized and acetylated waxy corn starch. Examples of pregelatinized and acetylated waxy starches are “Pregeflo® CH” marketed by Roquette, such as Pregeflo® CH10, CH20, CH30 or CH40.

Starchy Emulsifier or Emulsifier of Starchy Origin:

“Starchy emulsifier” is understood to mean a starch with emulsifying properties, in particular with the ability to emulsify an oil in water. A starchy emulsifier useful in the invention is thus a starch modified by a hydrophobic functionalization, or an amphiphilic functionalization, or an ionic functionalization, or a combination of these functionalizations. The starch undergoing at least one of said functionalizations can be a native starch, a pregelatinized starch, a hydrolyzed starch, a modified starch.

According to one embodiment, the starch undergoing at least one of said functionalizations is a native starch. According to another embodiment, the starch undergoing at least one of said functionalizations is a pregelatinized starch. According to another embodiment, the starch undergoing at least one of said functionalizations is a hydrolyzed starch.

“Emulsifier of starchy origin” is understood to mean a dextrin, or a hydrolyzed starch, or a maltodextrin, with the ability to emulsify an oil in water. An emulsifier of starchy origin is a dextrin, or a hydrolyzed starch, or a maltodextrin, having undergone a hydrophobic functionalization, or an amphiphilic functionalization, or an ionic functionalization, or a combination of these functionalizations.

Hydrophobic and/or Amphiphilic Functionalization

The term “hydrophobic and/or amphiphilic functionalization” denotes a chemical reaction between, on the one hand, a hydrophobic and/or amphiphilic reagent, and, on the other hand, some, or all, of the hydroxyl groups of the starch or of the matter of starchy origin. This reaction is generally a “substitution” or “grafting” by creating covalent bonds of the ester, ether or amide type.

According to an embodiment called “amphiphilic”, the starchy emulsifier, or the emulsifier of starchy origin, is obtained by substituting hydroxyl groups by reaction with an acid chloride, or with an alcohol and acid anhydride ester.

The acid chloride can be a chloride of one or more of the following acids, with from 2 to 24 carbons, preferentially from 4 to 24 saturated or unsaturated carbons, and more preferentially from among propionic acid, butyric acid, valeric acid, hexanoic acid, heptanoic acid, pelargonic acid, octanoic acid, decanoic acid, undecanoic acid, lauric acid, myristic acid, palmitic acid, oleic acid, stearic acid, the anhydrides of these acids, the mixed anhydrides of these acids, and any mixtures of these products.

The alcohol can be a linear, branched, or cyclic alcohol, made up of a carbon skeleton having at least 2 carbon atoms. The alcohol can comprise at least one unsaturated bond, that is at least one carbon-carbon double bond. The alcohol can be a linear, branched, or cyclic fatty alcohol made up of a carbon skeleton having from 8 to 36 carbon atoms. The fatty alcohol can comprise at least one unsaturated bond. Examples of unsaturated fatty alcohols are octanol, nonanol, decanol, undecanol, dodecanol, tetradecanol, hexadecanol, octadecanol, docosanol, policosanol.

The acid anhydride can be an anhydride of one of the polycarboxylic acids described hereinafter.

The polycarboxylic acid can be a linear, branched, or cyclic polycarboxylic acid consisting of a carbon skeleton having at least 2 carbon atoms. The polycarboxylic acid can comprise at least one unsaturated bond, that is at least one carbon-carbon double bond, such as, for example, maleic acid, glutathione acid, fumaric acid. The polycarboxylic acid can also comprise at least one alcohol group attached to the carbon chain. The polycarboxylic acid can comprise at least two acid groups. According to one embodiment, the polycarboxylic acids are linear dicarboxylic acids carrying the acid groups at the ends of the carbon chain. Examples of linear dicarboxylic acids are ethanedioic acid (or oxalic acid), propanoic acid, butanedioic acid (or succinic acid), dihydroxybutanedioic acid (or tartaric acid) 2-hydroxybutanedioic acid (or malic acid), pentanedioic acid (or glutaric acid), hexanedioic acid (or adipic acid), tetrahydroxyhexanedioic acid (or saccharic acid), gluconic acid, heptanoic acid (or pimelic acid), octanedioic acid, nonanedioic acid, decanedioic acid (or sebacic acid).

According to one embodiment, the acid anhydride is a linear dicarboxylic acid anhydride. According to one embodiment, the acid anhydride is succinic anhydride.

According to one embodiment, the alcohol and acid anhydride ester is a fatty alcohol ester and succinic acid anhydride, such as octenylsuccinic anhydride, or dodecylsuccinic anhydride.

According to one embodiment, the alcohol and acid anhydride ester is an ester of a C3-C15 saturated fatty alcohol, preferentially C4-C12, and most preferentially C5-C10, and of a C2-C10 acid anhydride, preferentially C3-C9, and most preferentially C4-C8. According to an alternative embodiment of this embodiment, the fatty alcohol comprises at least one unsaturated bond, that is at least one carbon-carbon double bond, preferably at least two unsaturated bonds, and most preferably at least three unsaturated bonds.

The level of functionalization can result in solubility of the functionalized starch. If the solubility is insufficient, a pregelatinization treatment can be applied to the functionalized starch to make it sufficiently soluble.

According to one embodiment, the emulsifying starch is a waxy starch functionalized by an alkenyl succinate group, in particular octenyl succinate or dodecyl succinate. Examples of starches carrying octenyl succinate functional groups are Cleargum® CO 01 and CO 03 marketed by Roquette.

According to another embodiment, the emulsifier of starchy origin is a dextrin having undergone octenyl succinate functionalization, such as, for example, Cleargum® CO A1 marketed by Roquette.

According to an embodiment called “hydrophobic”, the emulsifying starch, or the emulsifier of starchy origin, is obtained by grafting purely hydrophobic groups by radical reaction, for example, as disclosed in the applicant's application EP 3180372.

Gum of Microbial Origin:

The term “gum of microbial origin” refers to gums derived from fermentation of bacteria, such as xanthans, gelanes, dextrans and scleroglucans, or fermentation of yeasts such as beta-glucans, or derived from the biological activity of fungi, in particular molds such as 1-3-beta-glucans. The gum of microbial origin can be an endopolysaccharide or exopolysaccharide (EPS), that is a polysaccharide present in certain microorganisms on their cell walls and capable of being released in a culture medium.

Xanthan gum is a heteropolysaccharide produced on an industrial scale by aerobic fermentation of the bacterium Xanthomonas campestris. Xanthan gums generally have a molecular weight ranging between 1,000,000 and 50,000,000 Da. Among the commercially available products, the following can be cited, for example: Xanthan Gum FNCS-PC produced by Jungbunzlauer International AG, Keltrol® CG-T produced by CP Kelco, Cosphaderm® X 17 produced by Cosphagec, Kahlgum 6673 FEE-Xanthan gum produced by KahlWax, Rhodicare® S and Rhodicare® XC produced by Solvay and VANZAN® NF-C produced by Vanderbilt Minerais, NOVAXAN™ produced by ADM, and Kelzan® and Keltrol® produced by CP-Kelco.

Gellan gum is an anionic linear heteropolyoside based on oligoside units made up of 4 oses (tetra-oside). D-glucose, L-rhamnose and D-glucuronic acid in 2:1:1 proportions are present in gellan gum in the form of monomeric elements. It is sold, for example, under the name KELCOGEL CG

LA by CP KELCO.

Dextran gum is a branched polymer of dextrose (glucose) with a very high molecular mass, the dextrans are found in the sticky materials produced by the growth of certain bacteria, such as Leuconostoc mesenteroides, on saccharose media. They are made up of D-glucosyl units mainly joined by (1,6)alpha bonds. A range of dextran is sold, for example, by Pharmacosmos.

Scleroglucan gum is a non-ionic branched homopolysaccharide, made up of beta-D glucan units. The molecules consist of a main linear chain formed by D-glucose units bound by (1,3)beta bonds and one in three of which is bound to a lateral D-glucose unit by a (1,6)beta bond. An example of scleroglucan gum is the AMIGEL product sold by ALBAN MULLER.

Beta-glucan gum is a polysaccharide entirely made up of D-glucose bonded by beta bonds. The bonds can be very diverse and of the (1,3)beta, (1,4)beta or (1,6)beta type. Thus, the beta-glucans form a diversified group of molecules, particularly present in the cell walls of baker's yeast, and certain fungi and bacteria. For example, the Beta Glucan AC-25 product by Kraeber & Co GmbH is known.

Arabinogalactan gum is a polysaccharide present in varying amounts in many fungi and bacteria.

According to one embodiment, the gum of microbial origin is a xanthan gum or a sceroglucan gum, preferably a xanthan gum.

Plant-Based Gums:

The term “plant-based gums” refers to gums derived from seeds, tubers or exudates, plants, and gums extracted from algae. In the present invention, this term excludes starches and the derivatives thereof. The gums derived from seeds include galactomannans, such as guar gum, locust bean gum, tara gum, cassia gum. The gums derived from tubers include glucomannans such as konjac gum. The gums derived from plant exudates include arabic gum, adragant gum, karaya gum. The gums extracted from algae include alginates, galactans such as agar and carrageenans.

The gums useful in the invention are gelling gums alone or associated with each other.

Seed-Derived Gums

Galactomannans are non-ionic polyosides extracted from the albumen of leguminous seeds for which they constitute the reserve carbohydrate. Galactomannans are macromolecules made up of a main chain of (1,4)beta-bonded D-mannopyranose units, carrying lateral branches made up of a single D-galactopyranose unit bonded in (1,6)alpha to the main chain. The various galactomannans are distinguished, on the one hand, by the proportion of alpha-D galactopyranose units present in the polymer, and, on the other hand, by significant differences in terms of the distribution of galactose units along the mannose chain. The mannose:galactose ratio (M:G) is of the order of 2 for guar gum, 3 for tara gum, 4 for locust bean gum, and 5 for cassia gum.

Guar gum is characterized by a mannose:galactose ratio of the order of 2:1. The galactose group is evenly distributed along the mannose chain. Non-ionic, non-modified guar gums are, for example, the products sold under the name Vidogum GH, Vidogum G and Vidocrem by Unipektin and under the name Jaguar by Rhodia, under the name Meypro® Guar by Danisco, and under the name Supercol® guar gum by Aqualon.

Locust bean gum is extracted from the seeds of the carob tree, Ceratonia siliqua. It is characterized by a mannose:galactose ratio of the order of 4:1. The non-modified locust bean gum that can be used in this invention is sold, for example, under the name of “Vidogum L” by Unipektin, under the name Grinsted® LBG by Danisco.

Tara gum is derived from the albumen of the seeds of a south American tree, Caesalpinia spinosa. It is also called locust bean gum of Peru. It is made up of a chain of mannose monomers ((1,4)beta-D-mannopyranose) branched from galactose bridges 1-6. It is more branched than locust bean gum and less branched than guar gum since the ratio between the mannose and the galactose is 3:1, instead of 4:1 for locust bean gum and 2:1 for guar gum. An example of tara gum is that sold, for example, under the name of “Vidogum SP” by Unipektin.

Cassia gum is a galactomannan type polyoside such as guar gum and tara gum but obtained from the seeds of plants of the genus Cassia and Senna. It consists of a linear chain of mannose monomers bonded together by a (1,4)beta type osidic bond, to which all the surrounding five units of mannose are attached, by a (1,6)alpha type osidic bond, a galactose unit that yields a ratio of 5:1 between the mannose and the galactose. Cosmetic grades are available, for example, from Altrafine Gums under the name Semi-refined Cassia Gum.

Gums Derived From Tubers

Glucomannans are polysaccharides with a high molecular weight (between 500,000 and 2,000,000 Da), made up of D-mannose and D-glucose units with branching approximately every 50 or 60 units. It is found in wood, but it is also the main component of konjac gum. Konjac (Amorphophallus konjac) is a plant from the Araceae family. The products that can be used according to the invention are sold, for example, under the name Propol® and Rheolex® by Shimizu.

Gum of Plant Exudates

Arabic gum is a highly branched polysaccharide acid that is in the form of mixtures of potassium, magnesium and calcium salts. The monomer elements of the free acid (arabic acid) are D-galactose, L-arabinose, L-rhamnose and D-glucuronic acid.

Adragant gum, also called tragacanth or dragon gum, is an exudate obtained from the dried mucilaginous sap of approximately twenty species of plants of the Astragalus genus. This gum is a complex mixture of several polysaccharides. The two main fractions are tragacanthin (which is a neutral arabinogalactan) representing from 60% to 70% by weight, and bassorin, also called “tragacanthic acid” (which is a glycanogalacturonic acid) representing from 30% to 40% by weight.

Arabinogalactan gum is most often derived from American larch (Larix occidentalis).

Karaya gum (or Sterculia gum) is a plant-based gum obtained from the exudate of the branches of the Sterculia, the karaya gum is a polyoside mainly made up of galactose, rhamnose and galacturonic acid and a small amount of glucuronic acid.

Gums Extracted From Algae

Within the meaning of the invention “alginates” is understood to mean alginic acid, alginic acid derivatives and alginic acid salts (alginates) or said derivatives. Alginic acid, which is a natural substance derived from brown algae or certain bacteria, is a polyuronic acid made up of 2 uronic acids bonded by glycosidic bonds (1,4):Beta-D-manuronic acid and Alpha-L-glucuronic acid. Preferably, alginate compounds with an average molecular mass by weight ranging from 10,000 to 1,000,000, preferably from 15,000 to 500,000, and more preferably from 20,000 to 250,000, are preferably used.

The alginate-based compounds suitable for the invention can be represented, for example, by the products sold under the name Protanal™ by FMC Biopolymer, under the name GRINDSTED® Alginate by Danisco, under the name KEVIICA ALGIN by KEVIICA, and under the names Manucol® and Manugel® by ISP.

Galactans of the carrageenans type are anionic polysaccharides forming the cell walls of various red algae (Rhodophyceae) belonging to the families of Gigartinacae, Hypneaceae, Furcellariaceae and Polyideaceae. These linear polymers, formed by disaccharide units, are made up of two D-galactopyranoses units alternately bonded by (1,3)alpha and (1,4)beta bonds. These are highly sulfated polysaccharides (20-50%) and the alpha-D-galactopyranosyl residues can be in 3,6-anhydro form. Depending on the number and the position of ester-sulfate groups on the repeat disaccharide of the molecule, several types of carrageenans are distinguished, namely: kappa-carrageenans, which have an ester-sulfate group, iota-carrageenans, which have two ester-sulfate groups and lambda-carrageenans, which have three ester-sulfate groups. The carrageenans are basically made up of potassium, sodium, magnesium, triethanolamine and/or calcium salts and sulfate esters of polysaccharides.

Carrageenans are particularly marketed by Seppic under the name Solagum®, by Gelymar under the name Carragel®, Carralact®, and Carrasol®, and by CP-Kelco under the name GENULACTA®, GENUGEL® and GENUVISCO.

Galactans of the agar type are galactose polysaccharides contained in the cell wall of some of these red algae species (rhodophyceae). They are formed by a polymer group, the basic skeleton of which is a (1,3)beta D-galactopyranose and (1,4)alpha L 3-6 anhydrogalactose chain, with these units repeating regularly and alternately. The differences within the family of agars are due to the presence or absence of methylated or carboxylated solvated groups. These hybrid structures are generally present as a variable percentage, depending on the algae species and the harvesting season. Agar-agar is a mixture of polysaccharides (agarose and agaropectin) with a high molecular mass, ranging between 40,000 and 300,000 Da. It is obtained by manufacturing algae extraction juices, generally by autoclaving, and by treating these juices, which include approximately 2% agar-agar, in order to extract this agar-agar.

The agar is, for example, produced by the B&V Agar Producers group, under the name Gold Agar, Agarite and Large Agar by Hispanagar, and under the names Agar-Agar, QSA (Quick Soluble Agar), and Puragar by Setexam.

Other Plant-Based Gums:

In addition to the plant-based gums described above, other plant-based gums can be used: psyllium gum, pectins, mannans, galactoglucomannans, xylans, glycosaminoglycans, such as hyaluronic acid.

Pectins are substances that are present in large amounts in the primary walls of dicotyledonous plants, and in particular in the plant walls of many fruits and legumes, mainly citrus fruits and apples. They are rhamnogalacturonic type polysaccharides characterized by an alpha-D-galacturonic acid skeleton and small amounts of alpha-L-rhamnose more or less branched mainly by galactose and arabinose. It can involve pectic acids with a degree of methylation of less than 5% (DM<5), of low methylated pectins with a degree of methylation of less than 50% (DM<50) or of highly methylated pectins with a degree of methylation of more than 50% (DM>50). By way of an example, the product sold under the brand name GENU pHresh™ DF Pectin by CP Kelco can be cited.

Xyloglucan is a hemicelluloses compound that has a glucose residue skeleton (Glc), onto which xylose (Xyl), galactose (Gal) and fucose (Fuc) residues are grafted; they are found in many primary plant walls.

Xylan is a main component of hemicelluloses, and the second most abundant natural polysaccharide after xyloglucan. Xylans are xyloses polymers that include glucuronoxylans (GX), which have a xylose residue skeleton, onto which glucuronic acid residues (GlcA) or its O-methylated derivative are grafted, arabinoxylans (AX), which have a xylose residue skeleton, onto which arabinose residues are grafted, glucuronoarabinoxylans (GAX), which have a xylose residue skeleton, onto which arabinose and glucuronic acid residues are grafted; the arabinoxylans and glucuronoarabinoxylans are found in the primary walls of monocots and finally non-substituted homoxylans.

Mannan is a polysaccharide mainly made up of mannose monomers and denotes a set of polysaccharides belonging to the family of hemicelluloses that form the wall of the plant cells. These are monosaccharides bonded by 1,4-beta bonds. They can be linear or even branched, forming chains with a length (or degree of polymerization) ranging between 100 and 3,000 units.

Glycosaminoglycans (GAG or glycoaminoglycans) are carbohydrate macromolecules forming important components of the extracellular matrices of connective tissues of plant or marine origin. They are long sulfated linear chains (non-branched polymers) (except hyaluronic acid), made up of the repetition of disaccharides: a base diholoside always containing a hexosamine (glucosamine (GlcN) or galactosamine (GalN)) and another ose (glucuronic acid (GlcA), iduronic acid (IdoA), galactose (Gal)). The glucosamine is either N-sulfated (GlcNS), or N-acetylated (GlcNac). The galactosamine is always N-acetylated (GalNac). Hyaluronic acid, the derivatives and salts thereof can be cited from among the GAGs. These types of macromolecules are sold, for example, under the names MDI Complex® by Lucas Meyer Cosmetics, D-Factor by Res Pharma Industriale, Hydrocan by Tri-K Industries, Inc., Hyaluronic acid-BT by DSM Nutritional Products Europe Ltd.

Emulsion For Use in Cosmetics:

The oil-in-water type emulsion that is the object of the present application comprises:
at least one starchy emulsifier or emulsifier of starchy origin,
at least one thickening starch,
at least one gum of microbial origin,
at least two plant-based gums,
and at least one oil.

According to one embodiment, the oil-in-water emulsion comprises, or consists of:
at least one starchy emulsifier or emulsifier of starchy origin,
at least one thickening starch selected from among starches functionalized by crosslinking and stabilized, pregelatinized starches functionalized by crosslinking and stabilized, preferably from among crosslinked and acetylated starches, most preferably from among crosslinked and acetylated pregelatinized starches,
at least one gum of microbial origin,
at least two plant-based gums, at least including guar gum and tara gum,
at least one oil.

According to another embodiment, the oil-in-water emulsion comprises, or consists of:
at least one starchy emulsifier or emulsifier of starchy origin,
at least one thickening starch selected from among starches functionalized by crosslinking and stabilized, pregelatinized starches functionalized by crosslinking and stabilized, preferably from among crosslinked and acetylated starches, most preferably from among crosslinked and acetylated pregelatinized starches,
at least one gum of microbial origin,
a guar gum and a tara gum as sole plant-based gums,
at least one oil.

According to another embodiment, the oil-in-water emulsion comprises, or consists of:
at least one starchy emulsifier or emulsifier of starchy origin,
at least one thickening starch selected from among non-crosslinked, stabilized starches, non-crosslinked, stabilized pregelatinized starches, preferentially from among non-crosslinked acetylated starches, most preferentially from among non-crosslinked pregelatinized acetylated starches,
at least one gum of microbial origin,
at least two plant-based gums,
at least one oil.

According to another embodiment, the oil-in-water emulsion comprises, or consists of:
at least one starchy emulsifier or emulsifier of starchy origin,
at least one thickening starch selected from among non-crosslinked, stabilized starches, non-crosslinked, stabilized pregelatinized starches, preferentially from among non-crosslinked acetylated starches, most preferentially from among non-crosslinked pregelatinized acetylated starches,
at least one gum of microbial origin,
a guar gum and a tara gum as sole plant-based gums,
at least one oil.

According to another embodiment, the oil-in-water emulsion comprises, or includes as a single emulsifier, at least one starchy emulsifier or emulsifier of starchy origin selected from among a granular octenyl succinate starch, or a dextrin octenyl succinate, or an octenyl succinate modified gelatinized starch, or an octenyl succinate modified maltodextrin, or a mixture thereof. Preferentially, the starchy emulsifier is an octenyl succinate starch. According to another embodiment, the mass proportion of said at least one starchy emulsifier or emulsifier of starchy origin ranges from 0.20% to 3.60%, relative to the total weight of said oil-in-water emulsion.

According to another embodiment, the oil-in-water emulsion comprises, or includes as a single thickener, at least one thickening starch selected from among stabilized starches, preferentially acetylated starches, hydroxypropylated starches, hydroxyethylated starches, or more preferentially from among pregelatinized and acetylated starches, or pregelatinized and hydroxypropylated starches, most preferentially from among pregelatinized and acetylated starches, or the mixtures thereof. The thickening starch particularly can be a crosslinked and acetylated pregelatinized starch, or a non-crosslinked acetylated pregelatinized starch. According to another embodiment, the mass proportion of said at least one thickening starch ranges from 0.20 to 3.60%, relative to the total weight of the emulsion.

According to another embodiment, the oil-in-water emulsion comprises at least one gum of microbial origin selected from among xanthan gum, gellan gum, dextran gum, scleroglucan gum, beta-glucan gum, or the derivatives and mixtures thereof. According to one embodiment, the mass proportion of said gum of microbial origin ranges from 0.005% to 0.600%, relative to the total weight of the emulsion.

According to another embodiment, the oil-in-water emulsion comprises at least two plant-based gums selected from among galactomannans, glucomannans, galactans, alginates, preferentially from among guar gum, tara gum, locust bean gum, cassia gum, fenugreek gum, konjac gum, arabic gum, adragant gum, karaya gum, and most preferentially are guar gum and tara gum. According to one embodiment, the mass proportion of said at least two plant-based gums ranges from 0.06% to 2.700%, relative to the total weight of the emulsion.

According to one embodiment, said at least two plant-based gums are guar gum and tara gum, which are therefore the only plant-based gums present in the solid composition. According to one embodiment, the mass proportion of the guar gum ranges from 0.05% to 1.800%, and the mass proportion of the tara gum ranges from 0.010% to 0.900%, relative to the total weight of the emulsion.

According to one embodiment, the oil-in-water emulsion that is the object of the present application comprises:
from 0.20% to 3.60% of starchy emulsifier or emulsifier of starchy origin,
from 0.20% to 3.60% of thickening starch,
from 0.005% to 0.600% of gum of microbial origin,
from 0.015% to 2.700% of plant-based gums,
from 1% to 70% of oil, or from 10% to 60%, or from 15% to 50%, relative to the total weight of said emulsion.

According to one embodiment, the oil-in-water emulsion that is the object of the present application comprises:
from 0.40% to 3.00% of starchy emulsifier or emulsifier of starchy origin,
from 0.40% to 3.00% of thickening starch,
from 0.010% to 0.500% of gum of microbial origin,
from 0.120% to 2.250% of plant-based gums,
from 1% to 70% of oil, or from 10% to 60%, or from 15% to 50%, relative to the total weight of said emulsion.

According to one embodiment, the oil-in-water emulsion that is the object of the present application comprises:
from 0.60% to 1.80% of starchy emulsifier or emulsifier of starchy origin,
from 0.60% to 1.80% of thickening starch,
from 0.015% to 0.300% of gum of microbial origin,
from 0.180% to 1.350% of gums of plant origin,
from 1% to 70% of oil, or from 10% to 60%, or from 15% to 50%, relative to the total weight of said emulsion.

The oil-in-water emulsion comprises an oil selected from among polar non-volatile hydrocarbon oils, apolar non-volatile hydrocarbon oils, volatile oils, waxes, butters.

According to one embodiment, the oil-in-water emulsion comprises an oil selected from among silicone oils, hydrocarbon oils, ester oils, plant-based oils, preferentially from among ester oils and plant-based oils.

According to one embodiment, the mass proportion of oil in said emulsion ranges from 0.5% to 75%, or from 1% to 70%, or from 4% to 65%, or from 5% to 60%, or from 10% to 30%, by weight relative to the total weight of said emulsion.

According to one embodiment, the oil-in-water emulsion comprises less than 1% of at least one other emulsifier, preferentially less than 1% of another surfactant, in particular an ethoxylated surfactant, or of a low-biodegradable or non-biodegradable surfactant, preferentially less than 0.5%, or less than 0.01%, relative to the total weight of the emulsion.

According to one embodiment, the oil-in-water emulsion comprises:
at least one cosmetic additive selected from among polyols, organic acids, cationic or anionic polymers, fragrances, foaming surfactants, exfoliating agents, film-forming agents, preservatives, pigments, mineral fillers.
and/or at least one cosmetic active product selected from among moisturizing agents, anti-ageing products, UV filters, active ingredients extracted from plants.

According to one embodiment, the oil-in-water emulsion does not comprise a monosaccharide, and preferably no fructose. According to one embodiment, the oil-in-water emulsion does not comprise glucose-fructose syrup, also called high-fructose content corn syrup.

According to one embodiment, the oil-in-water emulsion consists of:
at least one starchy emulsifier or emulsifier of starchy origin,
at least one thickening starch,
at least one gum of microbial origin,
at least two plant-based gums,
at least one oil,
at least one cosmetic additive selected from among polyols, organic acids, cationic or anionic polymers, fragrances, foaming surfactants, exfoliating agents, film-forming agents, preservatives, pigments, mineral fillers.
at least one cosmetic active product selected from among moisturizing agents, anti-ageing products, UV filters, active ingredients extracted from plants.

According to one embodiment, the oil-in-water emulsion comprises an ingredient for use in cosmetics selected from among cationic surfactants, cationic polymers, pigments.

Oil

“Oil” is understood to mean any fatty substance in liquid form at room temperature (25° C.) and at atmospheric pressure (1,013,105 Pa).

Non-Volatile Oils

As previously indicated, the oil-in-water emulsion according to the invention comprises at least one non-volatile oil. More specifically, the non-volatile oil is selected from among silicone non-volatile oils, from among polar or apolar non-volatile hydrocarbon oils, and the mixtures thereof; and preferably from among polar non-volatile oils, in particular selected from among C10-C26 alcohols, ester oils, plant-based oils, alone or in mixtures.

“Hydrocarbon oil” is understood to mean an oil basically formed by, or even made up of, carbon and hydrogen atoms, and optionally of oxygen, nitrogen atoms, and not containing a silicon or fluorine atom. Hydrocarbon oil is therefore distinct from a silicone oil and a fluorinated oil. Within the meaning of the invention, “silicone oil” is understood to mean an oil comprising at least one silicon atom, and in particular at least one Si—O group. Non-volatile denotes oils with a vapor pressure that is less than 2.66 Pa, preferably less than 0.13 Pa (measurement according to OECD standard 104 dated 27 Jul. 95).

Polar Non-Volatile Hydrocarbon Oils

Preferably, the oil-in-water emulsion according to the invention comprises at least one non-volatile polar hydrocarbon oil. This hydrocarbon oil can contain alcohol, ester, ether, carboxylic acid, amine and/or amide groups. Preferably, the hydrocarbon oil is free of heteroatoms such as nitrogen, sulfur and phosphorus. In the present case, the polar non-volatile hydrocarbon oil comprises at least one oxygen atom. In particular, this polar non-volatile hydrocarbon oil comprises at least one alcohol functional group (it is then an “alcohol oil”) or at least one ester functional group (it is then an “ester oil”). The ester oils that can be used in the oil-in-water emulsion according to the invention in particular can be hydroxylated. Thus, the oil-in-water emulsion comprises one or more polar non-volatile hydrocarbon oil(s), in particular selected from among:
(1) C10-C26 alcohols, preferably monoalcohols: C10-C26 alcohols are saturated or unsaturated, branched or unbranched, and include from 10 to 26 carbon atoms, preferably from 14 to 24 carbon atoms. By way of examples of fatty alcohols that can be used according to the invention, linear or branched fatty alcohols can be cited, of synthetic or even natural origin, such as, for example, alcohols originating from plant-based (coprah, palm kernel, palm, etc.) or animal (tallow, etc.) materials. Of course, other long chain alcohols also can be used, such as, for example, ether alcohols or even alcohols called Guerbet alcohols. Finally, some longer or shorter cuts of alcohols of natural origin also can be used, such as, for example, coco (C12 to C16) or tallow (C16 to C18) or diol or cholesterol type compounds. By way of particular examples of fatty alcohols to be preferably used, lauric, isostearylic, oleic alcohol, 2-butyloctanol, 2-undecyl pentadecanol, 2-hexyldecyl alcohol, isocetyl alcohol, octyldodecanol and the mixtures thereof can be especially cited. According to an advantageous embodiment of the invention, the alcohol is selected from among octyldodecanol.
(2) the monoesters, diesters, triesters, optionally hydroxylated, of a C2-C8 mon- or polycarboxylic acid and a C2-C8 alcohol. In particular:
(2.1) the monoesters of a C2-C8 carboxylic acid and of a C2-C8 alcohol, optionally hydroxylated,
(2.2) the diesters of a C2-C8 carboxylic diacid and of a C2-C8 alcohol, optionally hydroxylated; such as diisopropyl adipate, diethyl-2 hexyl adipate, dibutyl adipate, 2-diethyl-hexyl succinate,
(2.3) the triesters of a C2-C8 carboxylic acid and of a C2-C8 alcohol, optionally hydroxylated, such as citric acid esters, such as trioctyl citrate, triethylcitrate, acetyltributyl citrate, tributyl citrate.
(3) the esters of a C2-C8 polyol and one or more C2-C8 carboxylic acids: such as glycol and monoacid diesters, such as neopentyl glycol diheptanoate, or glycol and monoacid triesters such as triacetin.
(4) ester oils, in particular having between 17 and 70 carbon atoms: by way of examples, mono -, di- or tri-esters can be cited. The ester oils may or may not be hydroxylated. The non-volatile ester oil can be selected, for example, from among:
(4.1) monoesters comprising between 17 and 40 carbon atoms in total, in particular the monoesters of formula R1-COO—R2, wherein R1 represents the residue of a linear or branched or aromatic fatty acid comprising from 4 to 40 saturated or unsaturated carbon atoms, and R2 represents a particularly branched hydrocarbon chain containing from 3 to 40 carbon atoms provided that R1+R2 are greater than or equal to 17, such as, for example, Purcellin (ketoaryl octanoate) oil, isononyl isononanoate, C12-C15 alcohol benzoate, ethyl 2-hexyl palmitate, octyldodecyl neopentanoate, octyl-2 dodecyl stearate, octyl-2 dodecyl erucate, isostearyl isostearate, octyl-2 dodecyl benzoate, octanoates, decanoates or ricinoleates of alcohols or polyalcohols, isopropyl myristate, isopropyl palmitate, butyl stearate, hexyl laurate, 2-ethyl hexyl palmitate, 2-hexyl-decyl laurate, 2-octyl decyl palmitate, 2-octyldodecyl myristate. Preferably, esters of formula R1-COO—R2 are involved, wherein R1 represents the residue of a linear or branched fatty acid having from 4 to 40 carbon atoms and R2 represents a particularly branched hydrocarbon chain containing from 3 to 40 carbon atoms, with R1 and R2 being 10 such that R1+R2 is greater than or equal to 17. Even more specifically, the ester comprises between 17 and 40 carbon atoms in total. By way of preferred monoesters, isononyl isononanoate, isopropyl palmitate, oleyl erucate and/or octyl-2-dodecenyl neopentanoate can be cited.
(4.2) fatty acid monoesters, in particular with 18 to 22 carbon atoms, and especially oleic acid, lauric acid, stearic acid, and diols, such as propylene glycol monostearate.
(4.3) diesters, in particular comprising between 18 and 60 carbon atoms in total, in particular between 18 and 50 carbon atoms in total. In particular, the following can be used: diesters of carboxylic diacid and monoalcohols, preferably such as diisostearyl malate; or the diesters of monocarboxylic acid and dialcohols, such as the 1,3-propanediyl ester of octanoic acid (or propanediol dicaprylate), sold under the name DUB ZENOAT by Stéarinerie Dubois; or the diesters of glycol and monocarboxylic acids, such as neopentyl glycol diheptanoate, propylene glycol dioctanoate, diethylene glycol diisononanoate, or polyglyceryl-2 diisostearate (especially such as the compound sold under commercial reference DERMOL DGDIS by Alzo);
(4.4) hydroxylated monoesters and diesters, preferably having a total carbon number ranging from 18 to 70, such as polyglyceryl-3 diisostearate, isostearyl lactate, octylhydroxystearate, octyldodecyl 1′hydroxystearate, diisostearyl malate, glycerin stearate;
(4.5) triesters, in particular comprising between 35 and 70 carbon atoms in total, in particular such as the triesters of tricarboxylic acid, such as triisostearyl citrate, or tridecyl trimellitate, or the triesters of glycol and monocarboxylic acids such as polyglycerol-2 triisostearate;
(4.6) tetraesters, in particular having a total carbon number ranging from 35 to 70, such as penthaerythritol tetraesters or polyglycerol and a carboxylic monoacid, for example, such as penthaerythrityl tetrapeptide, tetraisostearate penthaerythrityl, penthaerythrityl tetradecanoate, glyceryl tridecyl-2 tetradecanoate, polyglyceryl-2 tetraisostearate or even pentadeerythrityl tetradecyl-2 tetradecanoate;
(4.7) polyesters obtained by condensation of unsaturated fatty acid dimer and/or trimer and diol such as those described in patent application FR 0853634, such as, in particular, dilinoleic acid and 1,4-butanediol. In particular, in this respect the polymer marketed by Biosynthis under the name Viscoplast 14436H (INCI name: dilinoleic acid/butanediol copolymer), or even the copolymers of polyols and diacid dimers, and their esters, such as Hailucent ISDA, can be cited;
(4.8) esters and polyesters of diol dimer and mono- or dicarboxylic acid, such as esters of diol dimer and fatty acid and esters of diol dimer and dicarboxylic acid dimer, in particular that can be obtained from a carboxylic diacid dimer particularly derived from the dimerization of a C8 to C34 unsaturated fatty acid, notably C12 to C22, in particular C16 to C20, and more specifically C18, such as esters of dilinoleic diacids and dilinoleic diol dimers, for example, such as those marketed by NIPPON FINE CHEMICAL under the trade name LUSPLAN DD-DA5® and DD-DA7®;
(4.9) polyesters resulting from the esterification of at least one triglyceride of hydroxylated carboxylic acid(s) by an aliphatic monocarboxylic acid and by an aliphatic dicarboxylic acid, optionally unsaturated such as the castor oil of succinic acid and isostearic acid marketed under the reference Zénigloss by Zénitech;
(4.10) plant-based hydrocarbon oils such as triglycerides of fatty acids (liquids at room temperature), in particular fatty acids having from 7 to 40 carbon atoms, such as triglycerides of heptanoic or octanoic acids, in particular, saturated triglycerides can be cited such as caprylic/capric triglyceride and the mixtures thereof, for example, such as that marketed under the reference Myritol 318 by Cognis, glyceryl triheptanoate, glycerin trioctanoate, C18-36 acid triglycerides, such as those marketed under the reference DUB TGI 24 marketed by Stéarineries Dubois), jojoba oil, macadamia oil, apricot kernel oil, as well as unsaturated triglycerides such as castor oil, olive oil, ximena oil, pracaxi oil; and other plant-based hydrocarbon oils such as Camellia Japan seed oil, avocado oil, camelid oil, hazelnut oil, tsubaki oil, cashew nut oil, argan oil, soybean oil, grape seed oil, sesame oil, corn oil, wheat germ oil, rapeseed oil, sunflower oil, cotton oil, peanut oil.
(4.11) and the mixtures thereof, such as, for example, oils made up of a mixture of C8-C10 fatty acid monoesters and C12-C18 fatty alcohols, such as MIGLYOL Coco 810 by IOI Oleo GmbH (INCI name: coconut-Caprin/Caprate).

In a particular embodiment of the invention, the oil-in-water emulsion does not include a plant-based oil.

In a particular embodiment of the invention, the oil-in-water emulsion does not include canola oil.

Preferably, the one or more polar non-volatile hydrocarbon oil(s) is/are selected from among C10-C26 monoalcohols, ester oils, and in particular monoesters comprising at least 17 carbon atoms in total, diesters, which may or may not be hydroxylated, comprising at least 18 carbon atoms in total, triesters, in particular having at least 35 carbon atoms, tetraesters, in particular having at least 35 carbon atoms, plant-based hydrocarbon oils, and the mixtures thereof.

Apolar Non-Volatile Hydrocarbon Oils

With respect to apolar non-volatile oils, paraffin oil, squalane, pentadecane, nonadecane, eicosane, isoeicosane, polybutenes, which may or may not be hydrogenated, hydrogenated or non-hydrogenated polyisoprenes, hydrogenated or non-hydrogenated polydecenes, decene/butene copolymers, polybutene/polyisobutene copolymers, and the mixtures thereof can be cited more specifically. An example of a mixture of apolar non-volatile hydrocarbon oils is Emogreen L15 marketed by Seppic, which is a mixture of C15-C19 alkanes.

Non-Volatile Silicone Oils

With respect to silicone non-volatile oils, non-volatile, non-phenylated silicone oils can be cited, for example, such as polydimethylsiloxanes, for example.

Phenylated silicone oils, such as, for example, diphenyl dimethicone, phenyl trimethicone, trimethylsiloxyl phenyl dimethicone, diphenylsiloxy phenyl trimethicone, trimethyl pentaphenyl trisiloxane, or tetramethyl tetraphenyl trisiloxane, and the mixtures thereof also can be cited. Advantageously, the non-volatile silicone oil does not comprise an oxyalkylenated group(s) of C2-C3 (oxyethylenated, oxypropylenated), nor of glycerolated group(s).

According to a particular embodiment of the invention, the non-volatile oil is selected from among polar non-volatile oils, in particular selected from among C10-C26 alcohols, ester oils, plant-based oils, alone or in mixtures. Thus, as previously indicated, the oil-in-water emulsion comprises at least one C10-C26 alcohol, preferably C14-C24. The mass percentage of non-volatile oils more specifically represents from 4 to 65% by weight, preferably from 5% to 60%, more preferably from 10% to 30% by weight, relative to the weight of the oil-in-water emulsion.

Volatile Oils

The oil-in-water emulsion according to the invention optionally can comprise at least one volatile oil. Within the meaning of the invention, “volatile oil” denotes oils particularly having a non-zero vapor pressure, at ambient temperature and atmospheric pressure, in particular having a vapor pressure ranging from 2.66 Pa to 40,000 Pa, in particular ranging from 2.66 Pa to 13,000 Pa, and more specifically ranging from 2.66 Pa to 1 300 Pa. The volatile oils can be hydrocarbon or silicone.

In particular, apolar volatile hydrocarbon oils having from 8 to 16 carbon atoms can be cited like C8-C16 branched alkanes such as C8-C16 iso-alkanes (also called isoparaffins), isododecane, isodecane, isohexadecane and, for example, oils sold under the trade names of Isopars or Permetyls. Preferably, the volatile hydrocarbon oil is selected from among volatile hydrocarbon oils having from 8 to 16 carbon atoms and the mixtures thereof, in particular from isododecane, isodecane, isohexadecane, and is isohexadecane in particular. Volatile linear alkanes also can be cited comprising from 8 to 16 carbon atoms, in particular from 10 to 15 carbon atoms, and more specifically from 11 to 13 carbon atoms, for example, such as n-dodecane (C12) and n-tetradecane (C14) sold by Sasol under references PARAFOL 12-97 and PARAFOL 14-97, respectively, as well as the mixtures thereof, the undecane-tridecane mixture, such as Cetiol Ultimate by BASF, the mixtures of n-undecane (CFI) and n-tridecane (C13) obtained in Examples 1 and 2 of application WO 2008/155059 by Cognis, and the mixtures thereof, as well as ethers having at most 16 carbon atoms, such as, for example, dicaprylylether.

As silicone volatile oils, linear silicone volatile oils can be cited such as hexamethyldisiloxane, octamethyltrisiloxane, decamethyltetrasiloxane, tetradecamethylhexasiloxane, hexamethylheptasiloxane and dodecatrimethyl-pentasiloxane. 1′hexamethylcyclotrisiloxane, octamethylcylotetrasiloxane, decamethylcyclopenta-siloxane and dodecabmethylcyclohexasiloxane can be cited as cyclic silicone volatile oils.

Advantageously, if included in the oil-in-water emulsion, the volatile oil(s) content ranges between 0.5 and 10% by weight, or between 1 and 5% by weight, relative to the weight of the oil-in-water emulsion.

Waxes:

The oil-in-water emulsion according to the invention optionally can comprise at least one silicone wax, or a polar or apolar hydrocarbon wax. The wax considered within the scope of the present invention is generally a solid lipophilic compound at room temperature (25° C.), with a reversible solid/liquid state change, having a melting point that is particularly greater than or equal to 30° C., more specifically greater than 45° C. Advantageously, the melting point is less than or equal to 90° C., more specifically less than or equal to 80° C., and preferably less than or equal to 70° C. The melting point of a solid fatty substance can be measured using a differential scanning calorimeter (DSC), for example, the calorimeter sold under the name “DSC Q100” by TA Instruments with the “TA Universal Analysis” software.

The measurement protocol is as follows: a solid fatty substance of approximately 5 mg is placed in an “aluminum hermetic capsule” crucible. The sample undergoes a first temperature increase ranging from 20° C. to 120° C., at a heating rate of 2° C./minute up to 80° C., then is left in the isotherm at 100° C. for 20 minutes, then is cooled from 120° C. to 0° C. at a cooling rate of 2° C./minute, and finally undergoes a second temperature increase ranging from 0° C. to 20° C. at a heating rate of 2° C./minute. The melting temperature value of the solid fatty substance is the value of the top of the most endothermic peak of the observed melting curve, representing the variation in the difference in absorbed power as a function of the temperature.

Polar Hydrocarbon Waxes

More specifically, the polar wax is selected from among ester hydrocarbon waxes, alcohol hydrocarbon waxes, silicone waxes, and the mixtures thereof. “Hydrocarbon wax” is understood to be a wax basically formed by, or even made up of, carbon and hydrogen atoms, and optionally of oxygen, nitrogen atoms, and that does not contain a silicon atom or fluorine. It can contain alcohol, ester, ether, carboxylic acid, amine and/or amide groups. According to the invention, “ester wax” is understood to mean a wax comprising at least one ester functional group. The ester waxes also can be hydroxylated. According to the invention, “alcohol wax” is understood to mean a wax comprising at least one alcohol functional group, in other words comprising at least one free hydroxyl group (OH). The additional alcohol wax particularly does not comprise an ester functional group. “Silicone wax” is understood to mean a wax comprising at least one silicon atom, and in particular comprising Si—O groups.

Ester Waxes:

In particular, the following can be used as ester wax:

i) the waxes of formula R1-COO—R2, where R1 and R2 represent linear, branched or cyclic aliphatic chains, the number of atoms of which varies from 10 to 50, which can contain a heteroatom in particular oxygen, and the melting point temperature of which varies from 30° C. to 120° C., preferably from 30° C. to 100° C. In particular, a C20-C40 alkyl (the alkyl group comprising from 20 to 40 carbon atoms) (hydroxystearyloxy) stearate can be used as an ester wax, alone or as a mixture or a C20-C40 alkyl stearate. Such waxes are particularly sold under the names “Kester Wax K 82 P®”, “Hydroxypolyester K 82 P®”, “Kester wax K 80 P®”, or “KESTER WAX K82H” by KOSTEER KEUNEN. Mixtures of esters of C14-C18 carboxylic acids and alcohols also can be used, such as the “Cetyl Ester Wax 814” products by KOSTEER KEUNEN, “SP Crodamol MS MBAL”, “Crodamol MS PA” by CRODA, “Miraceti” by LASERSON. A glycol and butylene glycol montanate (octacosanoate) also can be used such as the LICOWAX KPS FLAKES (INCI name: glycol montanate) wax sold by Clariant.

ii) the di-(trimethylol-1,1,1propane) tetrastearate, sold under the name Hest 2T4S® by HETERENE.

iii) the diester waxes of a carboxylic diacid of general formula R3—(—OCO—R4-COO—R5), where R3 and R5 are identical or different, preferably identical and represent a C4-C30 alkyl group (alkyl group comprising from 4 to 30 (35) carbon atoms) and R4 represents a linear or branched C4-C30 aliphatic group (alkyl group comprising from 4 to 30 carbon atoms) and which may contain one or more unsaturated bonds. Preferably, the C4-C30 aliphatic group is linear and unsaturated.

iv) the waxes obtained by catalytic hydrogenation of animal or plant-based oils, in particular having C8-C32 linear or branched fatty chains, for example, such as hydrogenated jojoba oil, hydrogenated sunflower oil, hydrogenated castor oil, hydrogenated coprah oil, and waxes obtained by hydrogenation of castor oil esterified with cetyl alcohol, such as those sold under the names of Phytowax ricin 16L64® and 22L73® by SOPHIM. Such waxes are described in application FR-A-2792190. The waxes sold under the name “PHYTOWAX Olive 18 L 57” can be cited as waxes obtained by hydrogenation of olive oil esterified with stearyl alcohol.

v) the waxes of animal or plant origin, such as beeswax, synthetic beeswax, carnauba wax, candelilla wax, lanolin wax, rice bran wax, Ouricury wax, Alfa wax, berry wax, shellac wax, cork fiber wax, sugar cane wax, Japan wax, sumac wax, montan wax, orange and lemon wax, Laurier wax, hydrogenated jojoba wax, sunflower wax, in particular refined.

vi) hydrocarbon, polyoxyalkylenated or polyglycerolated, natural or synthetic, waxes of animal or plant origin also can be cited; the number of oxyalkylenated units (C2-C4) can vary from 2 to 100, the number of glycerolated units can vary from 1 to 20. By way of examples, polyoxyethylenated beeswaxes can be cited, such as PEG-6 beeswax, PEG-8 beeswax; polyoxyethylenated carnauba waxes, such as PEG-12 carnauba; hydrogenated or non- hydrogenated, polyoxyethenated or polyoxypropylenated, lanolin waxes, such as PEG-30 lanolin, PEG-75 lanolin; PPG-5 lanolin wax glyceride; polyglycerolated beeswaxes, in particular polyglyceryl-3 Beeswax, the mixture of Acacia decurens/Jojoba/Sunflower Seed wax/polyglyceryl-3 Esters, polyglycerolated plant waxes such as mimosa, jojoba, sunflower waxes, and the mixtures thereof (Acacia decurrens/Jojoba/Sunflower Seed Wax Polyglyceryl-3 Esters).

vii) The waxes corresponding to the partial or total esters, preferably total, of a C16-C30 carboxylic acid, that is saturated, optionally hydroxylated, with glycerol. Total esters means that all the hydroxylated functional groups of the glycerol are esterified. By way of an example, trihydroxystearin (or glyceryl trihydroxystearate), tristearin (or glyceryl tristearate), tribehenin (or glyceryl tribehenate), alone or as a mixture, can be cited. Among suitable compounds, glycerol triesters and 12-hydroxystearic acid, or hydrogenated castor oil, can be cited, such as, for example, Thixcin R, Thixcin E, marketed by Elementis Specialties.

viii) and the mixtures thereof.

Alcohol Waxes

As an alcohol wax, alcohols, preferably linear, preferably saturated, can be cited comprising from 16 to 60 carbon atoms, the melting point of which ranges between 25° C. and 90° C. By way of examples of alcohol wax, stearic alcohol, cetyl alcohol, myristic alcohol, palmitic alcohol, behenic alcohol, erucic alcohol, arachidyl alcohol, or the mixtures thereof, can be cited.

Apolar Hydrocarbon Waxes

The oil-in-water emulsion optionally can comprise at least one additional wax selected from among apolar hydrocarbon waxes. Within the meaning of the present invention, “apolar hydrocarbon wax” is understood to mean a wax containing only carbon or hydrogen atoms in its structure. In other words, such a wax is free of other atoms, in particular heteroatoms such as, for example, nitrogen, oxygen, silicon. By way of an illustration of apolar waxes suitable for the invention, hydrocarbon waxes such as microcrystalline waxes, paraffin waxes, ozokerite, polymethylene waxes, polyethylene waxes, waxes obtained by Fischer-Tropsch synthesis, microwaxes, in particular polyethylene, can be cited in particular.

Silicone Waxes

For example, mixtures comprising a C30-45 type Alkyldimethylsilyl Polypropylsilsesquioxane (INCI name) compound can be cited as a silicone wax, for example, the Dow Corning SW-8005 C30 Resin Wax product marketed by Dow Corning. Mixtures comprising a compound of the C30-45 Alkyl Methionine (INCI name) type also can be cited, such as, for example, the Dow Corning® AMS-C30 Cosmetic Wax product. Silicone beeswax also can be cited. The oil-in-water emulsion according to the invention can comprise a wax content(s), preferably polar, preferably hydrocarbon, ranging between 0.5 and 10% by weight, or from 0.5 to 6% by weight, or from 1 to 4% by weight, relative to the weight of the composition.

Use in Cosmetics and Cosmetic Products

The solid composition that is the subject matter of the present application can be used to prepare an industrial, or food, or pharmaceutical, or dermatological, or cosmetic oil-in-water emulsion. Preferably, the solid composition allows an oil-in-water emulsion to be prepared, and more preferably an emulsion with a transformation texture.

According to one embodiment, the solid composition is used to prepare an oil-in-oil emulsion that is a cosmetic product, selected from among skin care products, or hair care or hair coloring products, or oral care products, hygiene products, or make-up products, or a perfume. Preferably, the solid composition allows an oil-in-water cosmetic emulsion to be prepared with a transformation texture.

Method For Preparing an Oil-In-Water Emulsion

The method for preparing an oil-in-water emulsion that is the subject matter of the present application comprises the steps of:
dispersing and/or dissolving a solid composition that is the subject matter of the present application in an aqueous phase,
emulsifying the previously obtained aqueous phase with an oil phase.

According to one embodiment, the method comprises an emulsification step that is carried out at a temperature ranging from 10° C. to 90° C., or at a temperature ranging from 15° C. to 50° C., or at a temperature ranging from 18° C. to 35° C., or at a temperature ranging from 18° C. to 25° C.

A person skilled in the art can use any emulsification technique, in particular:
dispersing the required amount of solid composition in water at 20° C. under stirring at 1,000 rpm for 15 min,
adding the mass of oil under stirring at 2,500-3,000 rpm for 2 minutes - then continuing stirring at 3,000 rpm for 30 minutes.

Benefits of the Solid Composition, and of the Emulsion Obtained Therewith, According to the Invention:

The solid composition that is the subject matter of the present application allows oil-in-water emulsions to be prepared with very diverse pot textures depending on the amount used in said emulsion. “Pot texture” is particularly understood to mean the appearance and viscosity in a container, for example, a pot or a flask, before application on the skin. Used at a low mass percentage, that is less than or equal to 2%, or 1%, relative to the total weight of emulsion, the solid composition gives the emulsion a fluid texture, and thus allows an emulsion to be prepared in the form of a milk. When used at a high mass percentage, that is greater than or equal to 4%, or 5%, relative to the total weight of emulsion, the solid composition gives the emulsion a thick texture, and thus allows a thick cream to be prepared. For intermediate mass percentages, ranging from 2% to 4%, the texture of the emulsion will be that of a slightly fluid, to slightly thick cream. Irrespective of the mass percentage used in the emulsion, the solid composition gives the emulsion a shiny appearance.

The solid composition that is the subject matter of the present application also allows an oil-in-water emulsion to be prepared that has a transformation texture. “Transformation texture” is understood to mean an emulsion that has a texture when applied to the skin, in particular subject to shear stress, that differs from the pot texture, in particular a more fluid texture, and/or a texture that is both aqueous and oily. When the pot texture is that of a thick cream, the texture obtained by spreading on the skin will become a fluid texture, and can also feel like a mixture of an aqueous texture and an oily texture.

Without being bound by a theory, the applicant considers that this transformation texture is enabled by a phenomenon called “quick-break” phenomenon, in other words quick breaking of the emulsion subject to shear stress on the skin, with the novelty being that this quick-break phenomenon occurs in water and in oil, that is the feeling on the skin is both that of an aqueous phase and that of an oily phase. The solid composition that is the subject matter of the present application thus has the advantage of being a composition of natural origin allowing oil-in-water emulsions to be prepared that exhibit a quick-break phenomenon in water and oil.

In addition, the oil-in-water emulsions prepared with the solid composition that is the subject matter of the present application easily and homogeneously spread over the skin, and after the oil-in-water emulsion has penetrated, the feeling of stickiness is low, or even absent. Thus, for emulsions comprising mass percentages in oil with a moderate to low average, the solid composition that is the subject matter of the application allows an emulsion to be prepared that yields a rich feel. An average mass percentage in oil is understood to mean a mass percentage ranging from 60% to 20%, or from 50% to 25%, relative to the total weight of the emulsion.

A low mass percentage in oil is understood to mean a mass percentage ranging from 20% to 1%, or from 15% to 2.5%, or from 10% to 5%, relative to the total weight of the emulsion.

The oil-in-water emulsions prepared with the solid composition that is the subject matter of the present application exhibit good compatibility with anionic surfactants, preservative agents, salts, ethanol and pigments. When these ingredients are present, the emulsion remains stable, and its texture remains unchanged.

### BRIEF DESCRIPTION OF THE FIGURES

Further features, details and advantages of the invention will become apparent upon reading the appended figures.

FIG. 1 represents an illustration of the transformation texture and of the “quick-break” phenomenon.

### EXAMPLES

### Example 1: Preparation of a Solid Composition According to the Invention

Solid compositions according to the invention are prepared by dry mixing the powders of Table 1 in the indicated mass proportions.

TABLE 1

| Ingredient | CS1 | CS2 | CS3 | CS4 |
|---|---|---|---|---|
| Pregeflo ® CH40 | 43 | 35 | 35 | 40 |
| (Roquette Frères) | | | | |
| Cleargum ® CO 01 | 38 | 35 | 44.5 | 35 |
| (Roquette Frères) | | | | |
| Xanthan gum (AGI) | 1 | 4 | 2.5 | 3.5 |
| Guar gum (AGI) | 10 | 20 | 15 | 17 |
| Tara gum (AGI) | 8 | 6 | 3 | 4.5 |

The Cleargum® CO 01 can be replaced by Cleargum® CO 03 and Cleargum ® CO A1 in the same amounts.

### Example 2: Preparation of Sunflower Oil Emulsions and Their Stability

Oil-in-water emulsions are prepared from sunflower oil and using the emulsifying solid composition CS1 of Example 1 in two mass proportions, 2% m and 5% m, relative to the total weight of emulsion, and for mass proportions in oil ranging from 10% m to 70% m, relative to the total weight of emulsion, according to the compositions of Table 2.

TABLE 2

| | | Emulsions 1 to 5 | Emulsions 6 to 10 |
|---|---|---|---|
| | Ingredient | % m | % m |
| Phase A | Demineralized water | q.s. 100 | q.s. 100 |
| Phase A | Solid emulsifying | 2 | 5 |
| | composition CS1 | | |
| Phase B | Sunflower oil | 10, 20, 35, 50, 70 | 10, 20, 35, 50, 70 |
| | (Helianthus annuus | | |
| | seed oil) | | |

In order to prepare each emulsion, the required amount of solid emulsifying composition CS1 is dispersed in the water mass that is required in total at 20° C. under stirring at 1,000 rpm for 15 min. Then the oil mass is added under stirring at 2,500-3,000 rpm for 2 minutes. The stirring is then continued at 3,000 rpm for 30 minutes. The emulsion is then allowed to stand at 20° C. for 48 hours.

The Brookfield viscosities are measured after the standing period of 48 hours. The results are presented in table 3.

TABLE 3

| % oil | Mass % of CS1 = 2% m | Mass % of CS1 = 5% m |
|---|---|---|
| 10% | Too low | 10,400 |
| 20% | 3,340 | 12,640 |
| 35% | 5,940 | 30,350 |
| 50% | 16,780 | 72,200 |
| 70% | 64,200 | Too high |

By virtue of the composition CS1, by varying the mass proportion of oil between 10% and 70%, emulsions can be prepared with viscosities ranging from low values, that is approximately 3,000 mPa·s, and thus being in the form of a fluid milk, up to high values, that is approximately 72,000 mPa·s, and then being in the form of a thick cream. Intermediate viscosity values are also accessible, for example, values of 12,000 to 16,000 mPa·s, yielding emulsions in the form of medium-thick fluid cream.

The emulsions are kept preserved at 20° C., and the viscosity is measured after one week and then after one month of preservation.

TABLE 4

| % oil | 48 hours | One week | One month |
|---|---|---|---|
| 20% | 3,340 | 2,810 | 2,800 |
| 35% | 5,940 | 5,570 | 5,000 |
| 50% | 16,780 | 15,850 | 15,000 |
| 70% | 64,200 | 40,350 | 40,000 |

TABLE 5

| % oil | 48 hours | One week | One month |
|---|---|---|---|
| 10% | 10,400 | 10,760 | 12,000 |
| 20% | 12,640 | 12,850 | 16,000 |
| 35% | 30,350 | 27,750 | 27,000 |
| 50% | 72,200 | 65,100 | 62,000 |

For the two mass percentage values of composition CS1 that are implemented, the viscosities of the emulsions that are obtained are stable over a duration of at least one month (unstable=+/−25% in variation between one week and one month)

### Example 3: Preparation of Emulsion With Different Types of Oils

Oil-in-water emulsions are produced with mass percentages in oil of 10%, 30% and 60% according to the protocol of Example 2, using a mass percentage of composition CS1 of 3%, and using a single oil per emulsion, for the various oils of Table 6.

TABLE 6

| Oil # | Trade name | INCI name | Type | Supplier |
|---|---|---|---|---|
| H1 | Sweet almond | Prunus amygdalus | Plant- | Cooper |
| | | dulcis oil | based | |
| H2 | Isopropyl palmitate | Isosopropyl plamitate | Ester | BASF |
| H3 | Cosmacol | Dicaprylyl ether | Ether | Sasol |
| H4 | Dow Corning 1501 | Cylopentasiloxane (and) | Silicone | Dow |
| | fluid | dimethicone | | Corning |
| H5 | Belsil DM 350 | Dimethicone 350 | Silicone | Wacker |
| H6 | Mirasil CM 5 | Cyclopentasiloxane | Silicone | Blue |
| | | | | Silicone |
| H7 | Paraffin oil | Paraffinum liquidum | Alkane | Cooper |
| H8 | Isohexadecane | Isohexadecane | Alkane | IMCD |

Each emulsion is then assessed using a Brookfield viscosity measurement (at 20° C. at 20 rpm for 1 minute), and by measuring the particle sizes using an optical microscope, and by assessing the color of the emulsion.

TABLE 7

| | Viscosity (mPa · s) for the | | | | |
|---|---|---|---|---|---|
| | percentage in oil (% m) | | | Particle size | Color of the |
| Oil # | 10% | 30% | 60% | (microns) | emulsion |
| H1 | 8,000 | 11,000 | 80,000 | From <10 to 20 | white |
| H2 | 6,800 | 11,400 | 85,000 | From <10 to 20 | white |
| H3 | 7,500 | 13,000 | 85,000 | From <10 to 20 | white |
| H4 | 7,800 | 12,000 | 54,000 | From 30 to 100 | white |
| H5 | 6,500 | 12,000 | 42,000 | From 10 to 20 | white |
| H6 | 7,500 | 10,500 | 44 500 | From 10 to 50 | white |
| H7 | 7,200 | 11,500 | 44,000 | From 10 to 30 | white |
| H8 | 6,800 | 10,200 | 54,000 | From 10 to 30 | white |

The composition CS1 allowed white emulsions to be obtained with all the types of oil that were tested, with viscosities ranging from approximately 6 500 to 8,000 mPa·s, corresponding to a fluid cream texture, at approximately 80,000-85,000 mPa·s, thus corresponding to a thick texture.

The Brookfield viscosity measurements (at 20° C. at 20 rpm for 1 minute) of the emulsions were continued at 22° C. for 3 months (Table 7 bis), and at 50° C. for 1 month (Table 7 ter).

TABLE 7 bis

| | Brookfield Viscosity (mPa · s) for the | | |
|---|---|---|---|
| | percentage in oil (% m) | | |
| Oil # | 10% | 30% | 60% |
| H1 | 5,000 | 9,040 | 65,000 |
| H2 | 4,700 | 8,700 | n.d. |
| H3 | 5,000 | 10,000 | 53,000 |
| H4 | 5,200 | 8,500 | 24,000 |
| H5 | 4,500 | 7,500 | 27,000 |
| H6 | 5,000 | 8,000 | 30,500 |
| H7 | 5,200 | 9,000 | 33,000 |
| H8 | 4,800 | 8,300 | 30,000 |

TABLE 7 ter

| | Brookfield Viscosity (mPa · s) for the | | |
|---|---|---|---|
| | percentage in oil (% m) | | |
| Oil # | 10% | 30% | 60% |
| H1 | 8,400 | 10,100 | 58,000 |
| H2 | 7,100 | 9,800 | 47,000 |
| H3 | 6,100 | 9,600 | 32,000 |
| H4 | 6,900 | 8,400 | 14,700 |
| H5 | n.d. | 8,400 | n.d. |
| H6 | 6,800 | 6,000 | n.d. |
| H7 | n.d. | 8,300 | n.d. |
| H8 | n.d. | 8,500 | n.d. |

For almond oil (H1), isopropyl palmitate (H2) and dicaprylyl ether (H3), the average variation of the Brookfield viscosities when stored at 22° C. and 50° C. is 19% for mass percentages in oil of 10% to 30%, and 38% for a mass percentage in oil of 60%.

For cyclopentasiloxane-dimethicone (H4), dimethicone 50 (H5), cyclopentasiloxane (H6), paraffin oil (H7) and isohexadenane (H8), the average variation of the Brookfield viscosities when stored at 22° C. and 50° C. is 27% for mass percentages in oil of 10% to 30%, and 44% for a mass percentage in oil of 60%.

### Example 4: Stability of the Emulsions as a Function of pH

Emulsions are prepared according to the protocol of Example 2, using the following mass percentages: 5% of composition CS1, 20% of “Helianthus annuus seed oil”, 75% of demineralized water. The pH is adjusted to a target value corresponding to the values shown in Table 8, ranging from 2.6 to 12, with citric acid in solution or diluted soda. The Brookfield viscosity at 20 rpm is measured after 24 hours, then 7 days, of storage at 22° C.

TABLE 8

| | Viscosity at 24 hours | Viscosity at 7 days |
|---|---|---|
| pH | (mPa · s) | (mPa · s) |
| 2.6 | 11,000 | 11,000 |
| 4.1 | 12,300 | 12,500 |
| 4.7 | 11,700 | 11,700 |
| 6.3 | 10,500 | 10,100 |
| 8.5 | 8,500 | 6,700-start of exudate |
| 9.8 | 4,500 | Broken emulsion |
| 12 | 3,700 | Broken emulsion |

For a pH range ranging from 2.6 to 7.5-8, the viscosities of the prepared emulsions are stable enough to characterize these emulsions as stable.

The stability of the Brookfield viscosity (at 20° C. at 20 rpm for 1 minute) was studied when stored at pH values of 4, 4.7 and 6.5 over durations of 48 hours and 3 months at 22° C., and 1 month at 50° C. (Table 8 bis).

TABLE 8 bis

| | Brookfield | Brookfield | Brookfield |
|---|---|---|---|
| | Viscosity at | Viscosity at | Viscosity at |
| | 48 hours at 22° C. | 3 months at 22° C. | 1 month at 50° C. |
| pH | (mPa · s) | (mPa · s) | (mPa · s) |
| 4 | 12,500 | 13,800 | 15,000 |
| 4.7 | 11,700 | 11,400 | 12,700 |
| 6.5 | 10,800 | 11,400 | 12,600 |

It can be seen that at pH values that are less than or equal to 6.5 and greater than or equal to 4, the emulsions have a very stable Brookfield viscosity when stored at 22° C. for 3 months and at 50° C. for 1 month.

### Example 5: Stability of the Emulsions as a Function of the Salt Content

According to the protocol of Example 2, three emulsions are prepared with 5% of the composition CS1, 20% of “Helianthus annuus seed oil” and 75% of demineralized water. One of the emulsions constitutes the control sample. Another is added with 2% of sodium chloride. The latter is added with 2% of calcium chloride. The emulsions that are obtained after being stored for 48 hours at 20° C. (Table 9), 3 months at 20° C. (Table 9 bis), and after 1 month at 50° C. (Table 10), are characterized by measuring the Brookfield viscosity (20° C., 20 rpm), by assessing the particle size using an optical microscope, and the color of the emulsions.

TABLE 9

| | Brookfield | | | |
|---|---|---|---|---|
| | Viscosity at 20° C. | Particle size | | Color of the |
| Emulsion | and 20 rpm | (microns) | pH | emulsion |
| Control | 12,500 | From 10 to 20 | 5 | white |
| 2% NaCl | 13,000 | From 10 to 20 | 5 | white |
| 2% | 13,500 | From 10 to 20 | 5 | white |
| CaCl2 | | | | |

TABLE 10

| | Brookfield | | | |
|---|---|---|---|---|
| | Viscosity at 20° C. | Particle size | | Color of the |
| Emulsion | and 20 rpm | (microns) | pH | emulsion |
| Control | 16,000 | From 10 to 20 | 4.7 | white |
| 2% NaCl | 10,000 | From 15 to 30 | 4.3 | white |
| 2% CaCl2 | 10,500 | From 10 to 20 | 4.3 | white |

The results of tables 9, 9 bis and 10 show that adding 2% of salt does not alter the ability to emulsify, nor the quality and the stability of the emulsions that are obtained.

TABLE 9 bis

| | Brookfield Viscosity |
|---|---|
| | at 3 months at 20° C. |
| Emulsion | (mPa · s) |
| Control | 12,200 |
| 2% NaCl | 10,000 |
| 2% CaCl2 | 10,200 |

### Example 6: Stability of the Emulsions as a Function of the Surfactant Content

According to the protocol of Example 2, four emulsions are prepared with 3% of the composition CS1, 35% of “Helianthus annuus seed oil”, between 0% and 20% of a surfactant mixture sold under the name “Texapon WW100” by BASF, and the “sufficient amount to reach 100%” of demineralized water. The emulsions obtained after storing for 48 hours at 20° C. (Table 11) are characterized by assessing the particle size using an optical microscope, and the color of the emulsions.

TABLE 11

| | Brookfield | | | |
|---|---|---|---|---|
| | Viscosity at | | | |
| | 20° C. and 20 | Particle size | | Color of the |
| Emulsion | rpm | (microns) | pH | emulsion |
| Control sample 0% | 11,000 | <10 | 4.7 | white |
| Texapon WW100 | | | | |
| 5% Texapon | 7,800 | <10 | 6.8 | white |
| WW100 | | | | |
| 10% Texapon | 6,700 | <10 | 6.8 | white |
| WW100 | | | | |
| 20% Texapon | 5,200 | <10 | 6.8 | white |
| WW100 | | | | |

The emulsions prepared with the composition CS1 exhibit good tolerance to the presence of the mixture of anionic and non-ionic surfactants. The viscosities are lowered but remain acceptable. Moreover, the emulsions remain stable.

TABLE 11 bis

| | Brookfield | Brookfield |
|---|---|---|
| | Viscosity at 3 | Viscosity at 1 |
| | months at | month at 50° C. |
| Emulsion | 20° C. (mPa · s) | (mPa · s) |
| Control sample 0% | 12,700 | 13,000 |
| Texapon WW100 | | |
| 5% Texapon | 8,200 | 10,000 |
| WW100 | | |
| 10% Texapon | 7,200 | 8,200 |
| WW100 | | |

Storage at 20° C. was continued up to a duration of 3 months, and storage at 50° C. for a duration of 1 month (Table 11 bis) was implemented. For mass percentages of Texapon WW100 of less than or equal to 10%, it can be seen that the Brookfield viscosity varies from 5% to 15% when stored at 20° C., which is a low variation, and from 18% to 28% at 50° C., which is a notable variation. The low viscosity variations observed at 20° C. do not affect the texture of the emulsions, which remains unchanged relative to its initial state. The more notable variations at 50° C. do not however affect the texture of the emulsions in a manner that is perceptible by the user.

### Example 7: Compatibility With Pigments

Coloring emulsions are prepared with the “Unipure Yellow LC 182 HLC” pigment by Sensient Cosmetic Technologies:
either by introducing a pigment into the oil, then using the protocol of Example 2,
or by preparing an emulsion according to the protocol of Example 2, and then dispersing the pigment in the emulsion.

The cream is then applied to the back of the hand in order to assess the spreading quality and the homogeneity of the coloring (Table 12).

TABLE 12

| | Quality of the | Quality of | Homogeneity of |
|---|---|---|---|
| Pigment introduction mode | cream | spreading | the color |
| Before emulsification, in oil | smooth | average | poor |
| After emulsification, in | smooth | Very good | Very good |
| emulsion | | | |

It can be seen that introducing the pigment into the pre-prepared emulsion yields better results: the coloring cream spreads better, and yields a more homogeneous color.

TABLE 13

| | Quality of the | Quality of | Homogeneity of |
|---|---|---|---|
| Pigment | cream | spreading | the color |
| Unipure Yellow LC 182 HLC | Good | Good | Good |
| (hydrophobic) | | | |
| ASL-Yellow LL-100P | Good | Good | Good |
| (hydrophobic) | | | |
| SMS-1 Red N°122 | Good | Good | Good |
| (amphiphilic) | | | |
| Covarine Yellow WN 1798 | Good | Average | Average |
| (hydrophile) | | | |

It can be seen that using a solid composition CS1 according to the invention for preparing coloring emulsions allows emulsions to be obtained that spread well and have good coloring homogeneity.

Good results are also obtained from the solid compositions CS2, CS3 or CS4.

Coloring emulsions were prepared with the solid composition CS1 and with different colorings at mass percentages of 10% or 20% (relative to the weight of the emulsion), and by introducing the pigment in different ways: either in water, or in oil, or at the end, in other words in the emulsion that is obtained. The Brookfield viscosity is measured after storage at 22° C. for 48 hours and 3 months, and after storage at 50° C. for 1 month. The results are presented in Tables 13 bis and 13 ter. The Brookfield viscosity measurement spindle is the

SP6 spindle at 20° C. at 20 rpm for 1 minute.

TABLE 13 bis

| | Brookfield | Brookfield | Brookfield |
|---|---|---|---|
| | Viscosity at | Viscosity at | Viscosity at |
| | 48 hours | 3 months | 1 month |
| Pigment/Introduction/ | at 22° C. | at 22° C. | at 50° C. |
| Supplier | (mPa · s) | (mPa · s) | (mPa · s) |
| Covarine Yellow 10%/at | 26,300 | 35,000 | 45,000 |
| the end/Sensient | | | |
| SMS-1 Red N°211P 10%/ | 38,000 | 30,000 | 29,500 |
| at the end/Daito Kasei | | | |
| Unipure LC/PHY 10%/ | 26,000 | 24,000 | 25,300 |
| mixture at the end/Sensient | | | |
| Unipure LC/PHY 10%/red | 18,000 | 19,500 | 24,500 |
| at the end/Sensient | | | |
| Unipure LC/PHY 20%/in | 18,400 | 17,000 | 27,500 |
| water/Sensient | | | |
| Unipure HC/HLC 10%/at | 25,000 | 23,300 | 24,000 |
| the end/Sensient | | | |
| Unipure HC/HLC 10%/in | 28,100 | 24,000 | 28,500 |
| oil/Sensient | | | |

It can be seen that the coloring emulsions with the pigments of Table 13 bis are stable and have Brookfield viscosities that vary over periods of 3 months at 22° C. and 1 month at 50° C., but without a perceptible impact on the texture.

TABLE 13 ter

| | Brookfield | Brookfield | Brookfield |
|---|---|---|---|
| | Viscosity at | Viscosity at | Viscosity at |
| | 48 hours | 3 months | 1 month |
| | at 22° C. | at 22° C. | at 50° C. |
| Pigment/Supplier | (mPa · s) | (mPa · s) | (mPa · s) |
| Unipure LC/SGP 20% | 100,000 | 90,000 | 26,000 |
| in water/Sensient | | | |
| Unipure LC 20% in | 100,000 | 90,000 | 26,000 |
| water/Sensient | | | |
| Unipure LC 20% in | 87,000 | 43,000 | 19,500 |
| oil/Sensient | | | |

It can be seen that the coloring emulsions with the pigments of Table 13 ter have a relatively stable Brookfield viscosity at 22° C., but which drops significantly at 50° C., providing a more fluid cream. However, these variations have little impact on the emulsion, which remains stable, and on the dispersion of the pigments, which remains homogeneous, and on spreading on the skin, which also remains homogeneous.

Example 8: Illustration of the Transformation Texture

A transformation texture cream is prepared by using the solid emulsifying and texturing composition that is the subject matter of the present application, according to the composition of Table 14, according to the following protocol.

TABLE 14

| Phase | Ingredient (INCI) | Trade name (supplier) | Wt % |
|---|---|---|---|
| A | Aqua | Demineralized water | q.s. 100 |
| | Solid composition CS1 | not applicable | 3.00 |
| B | Propanediol dicaprylate | Dub Zenoat (Stéarinerie | 18.00 |
| | | Dubois) | |
| | Camellia Japonica Seed Oil | Tsubaki oil (Jan Dekker) | 2.00 |
| | Undecane (and) Tridecane | Cetiol Ultimate (BASF) | 30.00 |

The solid composition CS1 is dispersed in water at 20° C. under stirring at 1,000 rpm with a deflocculating blade, until the solid composition is hydrated and thus becomes opalescent, which requires approximately 5 to 10 minutes. Independently, the ingredients of phase B are mixed at 20° C. Still at 20° C., phase B is slowly added to phase A, for approximately 1 to 2 minutes, while stirring at 2,000-3,000 rpm with the deflocculating blade, and then stirring for a further 10 minutes.

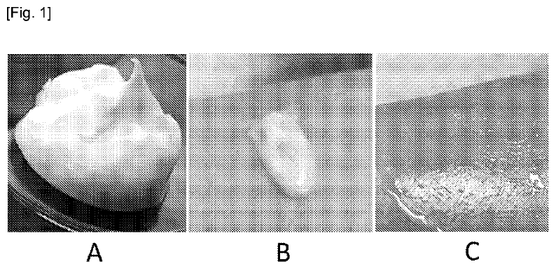

As illustrated in photograph A of FIG. 1, a thick, white “pot texture” cream is obtained, with a Brookfield viscosity, at 20° C. at 20 rpm with the SP6 spindle, from 23,000 to 27,000 mPas. This cream is stable for at least one month at 50° C. When the cream is taken and placed on the skin, the thick texture is maintained, as illustrated in photograph B of FIG. 1. Then, when the cream is spread on the skin, by making circular movements, which develop a shear force, the texture transforms into a mixture with an aqueous texture and an oily texture, as illustrated in photograph C of FIG. 1. This transformation appears to be the result of a phenomenon called “quick-break” phenomenon both in water and in oil.

According to the composition of Table 15, a variant of the previous cream with a transformation texture is prepared, by adding cosmetic additives, such as the isosorbide humectant sold under the name “Beauté by Roquette PO500” by Roquette Frères, paraben type preservatives, fragrance, and an anti-ageing cosmetic active ingredient, tocopherol, sold under the name of “Covi-ox T-70 C” by BASF.

TABLE 15

| Phase | Ingredient (INCI) | Trade name (supplier) | Wt % |
|---|---|---|---|
| A | Aqua | Demineralized water | q.s. 100 |
| | Isosorbide | Beauté by Roquette | 5.00 |
| | | PO500 (Roquette) | |
| | Solid composition CS1 | not applicable | 3.00 |
| B | Propanediol dicaprylate | Dub Zenoat (Stéarinerie | 18.00 |
| | | Dubois) | |
| | Camellia Japonica Seed Oil | Tsubaki oil (Jan Dekker) | 2.00 |
| | Undecane (and) Tridecane | Cetiol Ultimate (BASF) | 30.00 |
| | Phenoxyethanol• | Sepicide HB (Seppic) | 1.00 |
| | Methyl paraben• | | |
| | Ethylparaben Butylparaben | | |
| | Propylparaben | | |
| | Fragrance | Wild Bergamot (L.R. | 0.20 |
| | | Flavours & Fragrances) | |
| | Tocopherol | Covi-ox ® T-70 C | 0.10 |
| | | (BASF) | |

A thick cream is obtained as before, that is also stable, and also has a transformation texture with a quick-break phenomenon in water and in oil.

### Example 9: Compatibility With Ethanol

According to the protocol of Example 2, 4 emulsions are prepared comprising a mass percentage of 3% of composition CS1, 35% of “Helianthius annuus seed oil”, and “a sufficient amount to reach 100%” of demineralized water. One of the emulsions constitutes the control sample. Another is added with 5% by weight of ethanol relative to the total weight of the emulsion. The emulsions that are obtained after storage for 48 hours and 3 months at 20° C., and at the same time after 1 month at 50° C., are characterized by measuring the Brookfield viscosity (20° C., 20 rpm).

TABLE 16

| | Brookfield | Brookfield | Brookfield |
|---|---|---|---|
| | Viscosity at 48 | Viscosity at 3 | Viscosity at 1 |
| | hours at 22° C. | months at 22° C. | month at 50° C. |
| Emulsion | (mPa · s) | (mPa · s) | (mPa · s) |
| No ethanol | 11,000 | 12,700 | 13,000 |
| 5% ethanol | 6,200 | 7,000 | 9,000 |

It can be seen that ethanol can be added at a level of 5% by weight relative to the weight of the emulsion and a Brookfield viscosity can be maintained close to the initial viscosity, and that this viscosity is stable up to at least 3 months at 20° C. and 1 month at 50° C.

### Example 10: Compatibility With Preservatives

According to the protocol of Example 2, 4 emulsions are prepared comprising a mass percentage of 3% of composition CS1, 35% of “Helianthius annuus seed oil”, and “a sufficient amount to reach 100%” of demineralized water. One of the emulsions constitutes the control sample. The others are added with a preservative dose according to Table 17. The dose is expressed as a mass percentage, that is as a weight % of preservative relative to the total weight of the emulsion. The emulsions that are obtained after storage for 48 hours and 3 months at 20° C., and at the same time after 1 month at 50° C., are characterized by measuring the Brookfield viscosity at 20° C. and 20 rpm for 1 minute.

TABLE 17

| | Brookfield | Brookfield | Brookfield |
|---|---|---|---|
| | Viscosity at | Viscosity at | Viscosity at |
| Preservative and its dose | 48 hours | 3 months | 1 month |
| (% bv weight relative to | at 22° C. | at 22° C. | at 50° C. |
| the weight of emulsion | (mPa · s) | (mPa · s) | (mPa · s) |
| 0.5% Sodium benzoate + | 11,300 | 8,000 | 8,000 |
| 0.5% potassium sorbate | | | |
| 1% Caprylyl glycol | 9,000 | 7,400 | 6,300 |
| 1% Phenylpropanol & | 11,500 | 9,300 | 8,900 |
| 1,2-hexanediol | | | |
| 5% Pentylene glycol | 12,250 | 9,000 | 9,500 |

It can be seen that the emulsions prepared with the solid composition CS1 additivated with preservatives have a stable viscosity that drops slightly when stored at 22° C. for 3 months and at 50° C. for 1 month, but which remains sufficient to maintain the initial texture of the cream.

### Example 11: Compatibility With the Preparation Mode

Emulsions with 3% by weight of the solid composition CS1, 35% by weight of “Helianthus annuus seed oil” and 62% by weight of demineralized water, according to 5 different preparation modes, are prepared in order to assess the ease with which the emulsion can be prepared by means of a solid composition such as CS1:

“Deflocculator” preparation mode: this is a preparation protocol identical to that of Example 2, in which stirring is provided by a spindle of the “dispersing turbine” type, or even a “deflocculating turbine”.

“Conventional method” preparation mode: this is a preparation protocol identical to that of Example 2, in which stirring is provided by a spindle of the “marine propeller” type.

“Concentrated method” preparation mode: this is a preparation protocol similar to that of Example 2, but in which half the amount of total water required is used to produce the emulsion with the entire amount of oil required, in order to obtain a “concentrated emulsion”, and then half the amount of remaining water is added to the concentrated emulsion in order to dilute it and achieve the desired final composition.

“Rotor-stator” preparation mode: this is a preparation protocol identical to that of Example 2, in which stirring is provided by a rotor-stator type spindle.

“Ultra-turrax®” preparation mode: this is a preparation protocol identical to that of Example 2, in which stirring is provided by an “Ultra-turrax®” model rotor-stator manufactured by IKA.

The emulsions that are obtained after storage for 48 hours and 3 months at 20° C., and at the same time after 1 month at 50° C., are characterized by measuring the Brookfield viscosity at 20° C. and 20 rpm for 1 minute (Table 18)

TABLE 18

| | Brookfield | Brookfield | Brookfield |
|---|---|---|---|
| | Viscosity at 48 | Viscosity at 3 | Viscosity at 1 |
| | hours at 22° C. | months at 22° C. | month at 50° C. |
| Preparation mode | (mPa · s) | (mPa · s) | (mPa · s) |
| Deflocculator | 11,000 | 12,700 | 13,000 |
| Conventional method | 11,000 | 12,700 | 13,000 |
| Concentrated method | 12,000 | 10,700 | 12,000 |
| Rotor-stator | 11,600 | 10,300 | 12,500 |
| Ultra-turax | 12,800 | 14,500 | 11,400 |

It can be seen that the Brookfield viscosities of the emulsions prepared using all the tested preparation modes are stable.

### Example 12: Implementation in a Sunscreen Cream

A sunscreen cream was prepared by emulsification with a solid composition CS1 according to the composition of Table 19 following the protocol of Example 2, and by adding phase C to the emulsion that is obtained.

TABLE 19

| Phase | Ingredient (INCI) | Trade name (supplier) | Wt % |
|---|---|---|---|
| A | Aqua | Demineralized water | q.s. 100 |
| | Xanthan gum | Xantan Gum Personal care | 0.40 |
| | | grade | |
| | Solid composition CS1 | Solid composition CS1 | 4.00 |
| B | Camellia Japonica Seed | Tsubaki oil (Jan Dekker) | 23.00 |
| | Oil | | |
| | Homosalate | Eusolex HMS (Rona/Merck) | 5.00 |
| | Ethylhexyl | Eusolex 2292 (Rona/Merck) | 5.00 |
| | Methoxycinnamate | | |
| | Dietylamino | Uvinul A plus (BASF) | 4.00 |
| | Hydroxybenzoyl Hexyl | | |
| | Benzoate | | |
| | Bis-Ethylhexyl | Tinosorb S (BASF) | 13.00 |
| | Methoxyphenyl Triazine | | |
| C | Phenoxyethanol (and) | Microcare PHC (Thor) | 1.00 |
| | Chlorphenesin (and) | | |
| | Glycerin | | |

The sunscreen indices were determined using in-vitro protocols by the Helioscience laboratory according to the following protocol. Three “Sunplate” type PMMA plates were used, and 4 measurements were taken per plate. The cream prepared according to Table 19 was deposited on each plate. The plates underwent irradiation of 550 W/m2 for 30 minutes with an “ATLAS CPS+” solar simulator. Before and after irradiation, and before and after a water bath for water resistance, the level of photoprotection was measured with a “Kontron 933” spectrophotometer equipped with an integrating sphere. The results are presented in Table 20.

TABLE 20

| | SPF | Ery UVA | LOC (nm) | Water resistance (%) |
|---|---|---|---|---|
| Before irradiation | 85.9 | 36.8 | 375 | Not applicable |
| After irradiation | 83.2 | 36.1 | 374 | 69 |

The composition CS1 allowed a sunscreen cream to be prepared with a sun protection level of “50+”, and with water resistance of 69%.

1. A solid composition comprising:
at least one starchy emulsifier or emulsifier of starchy origin,
at least one thickening starch,
at least one gum of microbial origin,
and at least two plant-based gums.

2. The solid composition according to claim 1, wherein said at least one starchy emulsifier or emulsifier of starchy origin is a starch functionalized by at least one amphiphilic group selected from among an octenyl succinate granular starch, an octenyl succinate modified pregelatinized starch, an octenyl succinate modified gelatinized starch, an octenyl succinate functionalized dextrin, an octenyl succinate functionalized maltodextrin, or the mixtures thereof.

3. The solid composition according to claim 1, wherein said at least one thickening starch is selected from among stabilized starches, preferentially acetylated starches, hydroxypropylated starches, hydroxyethylated starches, or more preferentially from among pregelatinized and acetylated starches, or pregelatinized and hydroxypropylated starches, most preferentially from among pregelatinized and acetylated starches, or the mixtures thereof.

4. The solid composition according to claim 1, wherein said at least one gum of microbial origin is selected from among xanthan gum, gellan gum, dextran gum, scleroglucan gum, beta-glucan gum, or the derivatives and mixtures thereof.

5. The solid composition according to claim 1, wherein said at least two plant-based gums are selected from among galactomannans, glucomannans, galactans, alginates, preferentially from among guar gum, tara gum, locust bean gum, cassia gum, fenugreek gum, konjac gum, arabic gum, adragant gum, karaya gum, and most preferentially are guar gum and tara gum.

6. The solid composition according to claim 1, wherein the mass proportions, relative to the total weight of the composition, are:
from 20% to 60% of starchy emulsifier or emulsifier of starchy origin,
from 20% to 60% of thickening starch,
from 0.5% to 10% of gum of microbial origin,
from 2% to 45% of plant-based gums.

7. An oil-in-water type emulsion comprising:
at least one starchy emulsifier or emulsifier of starchy origin,
at least one thickening starch,
at least one gum of microbial origin,
at least two plant-based gums,
and at least one oil.

8. The emulsion according to claim 7, wherein said at least one starchy emulsifier or emulsifier of starchy origin is selected from among an octenyl succinate granular starch, an octenyl succinate dextrin, an octenyl succinate modified gelatinized starch, an octenyl succinate modified maltodextrin, or the mixtures thereof.

9. The emulsion according to claim 7, wherein the at least one starchy emulsifier or emulsifier of starchy origin is an octenyl succinate starch.

10. The emulsion according to claim 7, wherein said at least one thickening starch is selected from among stabilized starches, preferentially acetylated starches, hydroxypropylated starches, hydroxyethylated starches, or more preferentially from among pregelatinized and acetylated starches, or pregelatinized and hydroxypropylated starches, most preferentially from among pregelatinized and acetylated starches, or the mixtures thereof.

11. The emulsion according to claim 7, wherein said at least one gum of microbial origin is selected from among xanthan gum, gellan gum, dextran gum, scleroglucan gum, beta-glucan gum, or the derivatives and mixtures thereof.

12. The emulsion according to claim 7, wherein said at least two plant-based gums are selected from among galactomannans, glucomannans, galactans, alginates, preferentially from among guar gum, tara gum, locust bean gum, cassia gum, fenugreek gum, konjac gum, arabic gum, adragant gum, karaya gum, and most preferentially are guar gum and tara gum.

13. The emulsion according to claim 7, wherein said emulsion comprises an oil selected from among polar non-volatile hydrocarbon oils, apolar non-volatile hydrocarbon oils, volatile oils, waxes.

14. Use of a solid composition according to claim 1 for preparing an oil-in-water emulsion, preferentially an oil-in-water emulsion with a transformation texture.

15. The use of a solid composition according to claim 14, wherein the oil-in-oil emulsion is a skin care product, or a hair care or coloring product, or an oral care product, a hygiene product, or a makeup product, or a perfume.
